(19) United States
(12) Patent Application Publication
(10) Pub. No.: US 2003/0110522 A1
(43) Pub. Date: Jun. 12, 2003

(54) Generation of transgenic mouse models for the development of prostate cancer using regulatory regions of the PSP94 gene

(76) Inventors: Jim W. Xuan, London (CA); Joseph L. Chin, London (CA); Chandra J. Panchal, London (CA)

Correspondence Address: BROUILLETTE KOSIE PRINCE, 25th Floor, 1100 Rene-Levesque Blvd. West, Montreal, QC, H3B 5C9, CA

(21) Appl. No.: 10/231,773

(22) Filed: Aug. 30, 2002

(30) Foreign Application Priority Data
Aug. 31, 2001 (CA) 2,357,181

(51) Int. Cl. A01K 67/027; C12N 15/00; C12N 5/06

(52) U.S. Cl. 800/10; 800/18; 435/354; 435/320.1

(57) ABSTRACT

The utility of the promoter/enhancer region (i.e. regulatory region) of the PSP94 gene in targeting expression of heterologous genes, to the prostate gland was analyzed. Three breeding lines (i.e., line A, line B and line C) of the PSP-TGMAP model described herein were established with constructs containing regulatory regions with or without additional exon/intron sequence of the PSP94 gene. These regulatory regions were operatively linked to the simian virus 40 (SV40) T antigen (Tag, including both the large T and small t antigens). Histopathology evaluation of tumor grade, transgene expression, tumor responsiveness to androgen deprivation and tumor metastatic potential were analyzed and reported herein. This study indicates that regulatory regions (e.g., the promoter/enhancer with exon/intron region) of the PSP94 gene efficiently target the expression of heterologous genes to prostate tissue. Furthermore, the PSP-TGMAP model provides a valuable tool for the analysis of prostate cancer development and for developing new gene therapy vector for the treatment of prostate cancer. Based on this study, mouse PSP94 promoter/enhancer region may be used in the generation of a “shuttle vector” fit for prostate targeting in both mouse CaP model and in human clinical trial.

### FIELD OF THE INVENTION

This invention relates to DNA constructs and transgenic non-human mammals (e.g., animals). More particularly, transgenic mice carring a gene(s) (i.e., one or more copies of a gene) comprising regulatory regions (promoter/enhancer region coupled to exon and/or intron sequences) of PSP94 were developed herein. These PSP94 regulatory regions target the mouse prostate tissue and when operatively linked to a gene capable of initiating tumor formation, leads to prostate tumor development. Regulatory region of the PSP94 gene may be used in the development of gene therapy tools for the treatment of prostate cancer (CaP) in higher mammals.

### BACKGROUND OF THE INVENTION

The prostate gland, which is found exclusively in male mammals, produces several components of semen as well as several regulatory peptides. The prostate gland comprises stroma and epithelium cells, the latter group consisting of columnar secretory cells and basal nonsecretory cells. A proliferation of these basal cells as well as stroma cells gives rise to benign prostatic hyperplasia (BPH), which is one common prostate disease. Another common prostate disease is prostate cancer (CaP), which is the most common of the fatal pathophysiological prostate cancers, and involves a malignant transformation of epithelial cells in the peripheral region of the prostate gland. Prostate cancer and benign prostatic hyperplasia are two common prostate diseases, which have a high rate of incidence in the aging human male population. For example, prostate cancer is clinically diagnosed in approximately 10% of men during their lifetime (185,000 cases/year). It will claim 39,000 lives each year, which represents 13% of cancer related deaths in males.

Studies of the various substances synthesized and secreted by normal, benign and cancerous prostates were carried out in order to gain an understanding of the pathogenesis of prostate diseases. These studies revealed that proteins may be used as immunohistochemical tumor markers in the diagnosis and/or prognosis of prostate disease. The three predominant proteins secreted by a normal prostate gland are: [1] Prostatic Acid Phosphatase (PAP); [2] Prostate Specific Antigen (PSA); and, [3] Prostate Secretory Protein of 94 amino acids (PSP94), which is also known as Prostatic Inhibin Peptide (PIP), Human Seminal Plasma Inhibin (HSPI), or α-microseminoprotein (β-MSP), and which is hereinafter referred to as PSP94. U.S. Pat. No. 5,428,011, the entire content of wich is incorporated herein as reference, describes work performed on the PSP94 gene.

Both PSA and PAP have been studied as tumor markers in the detection of prostate disease, but since both exhibit elevated levels in prostates showing benign prostatic hyperplasia (BPH), neither marker is specific and therefore they are of limited utility.

A major therapy in the treatment of prostate cancer is androgen-ablation. While most patients respond initially to this treatment, its effectiveness decreases over time, possibly because of the presence of heterogenous populations of androgen-dependent and androgen-independent cells to the androgen treatment, where any androgen insensitive cells would continue to proliferate unabated. Therapies available to date such as surgical, hormonal and radiation therapy either alone or in combination does not appear to adequately control advanced prostate cancer. There is no cure for the majority of patients diagnosed with advanced disease. Some of them will eventually develop distant metastasis leading to a decrease in survival expectancy.

The development of prostate cancer is essentially restricted to humans. To date, the pathogenesis of CaP remains one of the most elusive. For example, the propensity of CaP to metastasize to bone and the emergence of androgen-independent forms of CaP have diminished previous expectations to comprehensively study this disease. Furthermore, the initiation of CaP from prostate intraepithelial neoplasia (PIN) and benign prostatic hyperplasia (BPH) is still a matter of debate. Researchers are now focusing at reducing the morbidity and mortality related to this pathology. New strategies directed toward the treatment of advanced or recurrent CaP are needed in order to achieve long-term local control of CaP. Alternative strategies include, for example, gene therapy. Lack of a proper animal model has been one of the most challenging issues to overcome. Mouse, for example, does not develop prostatic neoplasia spontaneously. For this purpose, development of animal models, mimicking the development of prostate cancer in humans, are anxiously awaited.

The mainstay of animal model development and gene therapy is the identification of tissue specific element(s) that will specifically target prostate tissue without affecting significantly other tissues. For clinical trial purpose, a vector that may be selected for prostate-specific targeting may possess, for example, [1] a part encoding a heterologous protein that is evolutionarily relatively conserved in both structure and function in order to be tested in animal models (pre-clinical trial) and human clinical trials, and [2] a prostate tissue specific element (TSE) that would be part of a strong promoter/enhancer region (i.e., a regulatory region).

Several regulatory regions (e.g., promoters) were tested in order to develop an animal model for prostate cancer studies. Among them some non-prostate specific regulatory regions, e.g., the mouse mammary tumor virus (MMTV) long terminal repeat (LTR), mouse cryptdin-2 (CR-2) regulatory region, human fetal γ-globin regulatory region, bovine keratin 5 regulatory region, and gp91-phox regulatory region (Kitsberg, D. I., and Leder, P., Oncogene, 13:2507-2515, 1996; Garabedian, E. M. et al., Proc. Natl. Acad. Sci. U.S.A., 62:227-237, 1998; Perez-Stable, C., et al., Cancer Res., 57:900-906, 1997; DiGiovanni, J. et al., Proc. Natl. Acad. Sci. U.S.A., 97:3455-3460, 2000; Blackburn, R. V., et al., Cancer Res., 58:1358-1362, 1998; for review see: Sharma, P., and Scheiber-Agus, N., Oncogene, 18:5349-5355, 1999). Some of the transgenic mice generated, rapidly developed progressive prostate cancers, even in the absence of androgen stimulation and also provided a potent model for neuroendocrine-derived prostate neoplasia. However none of these models showed specific targeting to the prostate gland (review see: Sharma, P., and Scheiber-Agus, N., Oncogene, 18:5349-5355, 1999; Green, J. E., et al., Prostate, 36:59-63, 1998). The gene of the rat prostate steroid-binding protein (PSBP or C3) was found to target the prostate gland as well as the mammary gland in transgenic mice (Maroulakou, I. G., et al., Proc. Natl. Acad. Sci. U.S.A., 91:11236-11240, 1994; Schibata, M. A. et al., Cancer Res., 56:4894-4903, 1996; Schibata, M. A. et al., EMBO J., 18:2692-2701, 1999).

Currently there are only a few prostate specific genes (e.g. prostate specific antigen (PSA), rat probasin (rPB)), having been tested for the development of animal models and gene therapy vectors. None of these showed the capacity to be used in the generation of a “shuttle vector” fit for prostate targeting in both mouse CaP model and in human clinical trial.

A transgenic adenocarcinoma mouse prostate (TRAMP) model was generated by using a short regulatory region derived from the rat probasin promoter in order to direct the expression of heterologous proteins (Gingrich, J. R., et al., Cancer Res., 56:4096-4102, 1996; Greenberg, N. M., et al., Proc. Natl. Acad. Sci. U.S.A., 92:3439-3443, 1995; Gingrich, J. R., et al., Cancer Res., 57:4687-4691, 1997). The use of a longer promoter (the LPB-Tag model) controlling the expression of the SV40 large T antigen was shown to target the expression to the prostate tissue and to induce CaP (Kasper, S., et al., Lab. Invest., 78:319-333, 1998; Zhang, J., et al., Endocrinology, 141:4698-4710, 2000). As compared to existing transgenic models of CaP, the TRAMP and LPB-Tag models show a number of advantages. The use of the rPB promoter (i.e., regulatory elements) leads to transgene expression as well as tumor-induced formation that are restricted to the prostate, mostly to the ventral prostate (VP) lobes and the dorsolateral prostate (DLP) lobes (Greenberg, N. M., et al., Proc. Natl. Acad. Sci. U.S.A., 92:3439-3443). Furthermore, metastatic tumors with close resemblance to human CaP were identified in these models (Gingrich, J. R., et al., Cancer Res., 56:4096-4102, Gingrich, J. R., et al., Cancer Res., 57:4687-4691, 1997, Masumori, N., et al., Cancer Res. 61:2239-2249, 2001).

The second prostate-specific gene; PSA has also been tested for prostate specific targeting. Several potential prostate tissue specific elements (TSE) from the PSA promoter/enhancer region were characterized by in vitro tissue cell culture experiments (Rodrigez, R., et al., Cancer Res., 57:2559-2563, 1997; Pang, S., et al., Cancer Res., 57:495-499, 1997; Schuur, E. R., et al., Biol. Chem., 271:7043-7051, 1996). In vivo, a 657 base pair (bp) of the human PSA gene promoter linked with T24-ras was found to induce tumor formation in the salivary gland and gastrointestinal tract of transgenic mice (Schaffner, D. L., et al., Lab. Invest. 72:283-290, 1995). Two other PSA transgenes were constructed based on a 6 kilobase (kb)-long regulatory region derived from human PSA promoter or the whole (12 kb) human PSA promoter (Cleutjens, K. B., et al., Mol. Endocrinol., 11:1256-1265, 1997; Wei, C., et al., Proc. Natl. Acad. Sci. U.S.A., 94:6369-6374, 1997). Those were reported to be essentially prostate-specific, although only a Lac Z reporter gene was used. PSA promoter region has not been used to target transgenes that disrupt prostatic function in transgenic mice. Because of difficulties in using long promoter/enhancer region of human PSA gene for constructing an adenovirus based gene therapy vector, current pre-clinical trials are performed without prostate targeting in animal model (Latham, J. P. F., et al., Cancer Res., 60:334-341, 2000; Yu, D. C., et al., Cancer Res. 59:1498-1504, 1999). For example, pre-clinical trials using a combination of rat probasin and human PSA promoter/enhancer regions were set up by using human LNCaP cell line xenograft tumors in nu/nu mouse and recombinant adenovirus. Elimination of tumor growth was observed (Rodrigez, R., et al., Cancer Res., 57:2559-2563, 1997; Yu, D. C., et al., Cancer Res., 61:517-525, 2001).

Results using both rat PB and human PSA regulatory region for targeting prostate tissue were challenged by the fact that neither a human counterpart of the rPB gene nor a rodent counterpart of human PSA has been clearly identified due to interspecies divergence. Neither rPB nor PSA regulatory regions can be used to generate “shuttle vectors” for use in animal models (i.e., testing in pre-clinical studies) and in human clinical studies. Identification of new prostate tissue specific elements (i.e., regulatory regions) is definitively needed to supplement the current studies on prostate targeting and gene therapy of CaP.

PSP94, another prostate-specific protein has been identified recently. Metabolic and immunohistochemical studies have shown that the prostate is a major source of PSP94. PSP94 is a simple non-glycosylated cysteine-rich protein, and constitutes one of three predominant proteins found in human seminal fluid along with Prostate Specific Antigen (PSA) and Prostate Acid Phosphatase (PAP). PSP94 has a molecular weight of 10.7 kDa. The cDNA and gene for PSP94 have been cloned and characterized (Ulvsback, et al., Biochem. Biophys. Res. Comm., 164:1310, 1989; Green, et al., Biochem. Biophys. Res. Comm., 167:1184, 1990). Immunochemical and in situ hybridization techniques have shown that PSP94 is expressed predominantly in prostate epithelial cells. It is also present, however, in a variety of other secretory epithelial cells (Weiber, et al., Am. J. Pathol., 137:593, 1990). PSP94 was also shown to be expressed in prostate adenocarcinoma cell line, LNCap (Yang, et al., J. Urol., 160:2240, 1998). An inhibitory effect of exogenous PSP94 on tumor cell growth has been observed both in vivo and in vitro, suggesting that PSP94 could be a negative regulator for prostate carcinoma growth via interaction with cognate receptors on tumor cells (Garde, et al., Prostate, 22:225, 1993; Lokeshwar, et al., Cancer Res., 53:4855, 1993). In addition, PSP94 has been shown to have a therapeutic modality in treating hormone refractory prostate cancer.

Both PSA and PSP94 have a similar tissue distribution, for example, these proteins can be found in breast tissue, in tracheal tissue, in gastric juice, and in saliva, (for reviewed see: Wu, D., et al., J. Cell. Biochem., 76:71-83, 1999; Imasato, Y., et al., Endocrinol., 142:2138-2146, 2001). The level of expression of both PSA and PSP94 in non-prostatic tissue is significantly low and minimal compared with the amount found in the prostate fluid and semen (106 to 109 lower). It is conceivable that the low non-prostate expression of PSP94 observed may be the result of a leak from the strong PSP94 promoter/enhancer (Dube, J. Y., et al., J. Androl. 8:182-189, 1987).

The invention disclosed herein relates to transgenic non-human mammals (e.g., mice) susceptible to prostate tumor formation. In this application, regulatory regions (promoter/enhancer with exon/intron region) of the mouse PSP94 gene, may be used to direct the specific expression of a heterologous protein (e.g., SV40 Tag) to the prostate of transgenic mice. The “shuttle vector” comprising PSP94 regulatory regions may be used, for example, to study the pathogenesis and to treat CaP by gene therapy approaches in animal and humans.

### SUMMARY OF THE INVENTION

In accordance with one aspect, the present invention relates to a DNA construct (e.g., A DNA fragment, vector, plasmid linearized or not) comprising a regulatory region of the mouse PSP94 gene. DNA constructs comprising such regulatory regions may be used for various applications. For example, these constructs may be used to generate a transgenic non-human mammal, especially a transgenic non-human mammal having a (foreign) gene expressed (mainly) in its prostate (tissue, cells). These DNA constructs may also be used to express a gene in isolated (prostate) cells (lines), for research purpose or therapeutic purpose (e.g., gene therapy).

In accordance with another aspect, the present invention relates to a DNA construct (e.g., A DNA fragment, vector, plasmid linearized or not) comprising a regulatory region of the mouse PSP94 gene operatively linked to a gene capable of initiating tumor formation.

In another aspect, the present invention relates to a DNA construct comprising the transgene 183, as described herein and illustrated in FIG. 1C.

In yet another aspect, the present invention relates to a DNA construct comprising the transgene 186 as described herein and illustrated in FIG. 1D.

In a further aspect, the present invention relates to a DNA construct comprising a regulatory region of the mouse PSP94 gene operatively linked to a gene encoding a therapeutic protein.

In accordance with the present invention, the therapeutic protein may be selected from the group consisting of a cytotoxic protein (acting directly or indirectly, i.e., by converting an inactive (i.e., inert) compound into an active (e.g. cytotoxic) compound)) (e.g., the A chain of diphteria toxin, ricin, abrin, etc.), a protein causing apoptosis (e.g., caspases (caspase-3, caspase-8), Fas-Ligand, Bax, TRAIL, etc.), an anti-oncoprotein (e.g., p53, p21, p20, Rb, etc.), a protease (e.g., awsin, papain, proteinase K, carboxypeptidase, etc.), a suicide protein (i.e., encoded by a suicide gene e.g., herpes simplex virus-1 thymidine kinase, Escherichia coli cytosine deaminase, etc.), a cytokine (e.g., IL-1, IL-2, IL-6, IL-12, GM-CSF, G-CSF, M-CSF, IFN-alpha, IFN-beta, IFN-gamma, TNF-alpha, TNF-beta, etc.), a chemokine (e.g., Mig-1alpha, Mig-1beta, IP-10, MCP-1, etc.) a costimulatory molecule and an antigen (e.g., a (prostate) tumor associated antigen).

It is known in the art that HSV-1 TK and/or cytosine deaminase renders the cells capable of metabolizing 5-fluorocytosine (5-FC) to the chemotherapeutic agent 5-fluorouracil (5-FU). HSV-1 TK can also phosphorylates gancyclovir (GCV) converting it into a non-diffusible nucleoside analogue that terminates DNA synthesis leading to cell death.

In an additional aspect, the present invention relates to a DNA construct comprising a regulatory region of the mouse PSP94 gene operatively linked to a gene encoding a reporter protein.

In accordance with the present invention, the reporter protein may be selected from the group consisting of beta-galactosidase, luciferase, red fluorescent protein, green fluorescent protein, alkaline phosphatase, chloramphenicol acetyl transferase, and horseradish peroxidase.

The gene chosen to be (operatively) linked with the regulatory regions described herein may also be a gene able to express a protein stimulating (helping) the immune system to eliminate (prostate) tissue (or cancer cells) expressing such protein. These may be antigens (e.g., PSA, PSP98, prostate specific membrane antigen (PSMA)), or other tumor-associated antigen) modified (mutated) or not and/or co-stimulatory molecules (e.g. B7.1) (Chambers, C. A. and J. P. Allison, Curr Opin. Cell Biol., 1999, 11(2):203-211; Forni, G. et al., Cytokines Mol. Ther. 1995, 1(4):225-248). The gene chosen to be (operatively) linked with the regulatory regions described herein may also be a gene able to direct the expression of an antisense RNA, a ribozyme that will target a desired gene (its mRNA) express in prostate tissue (for a general review on antisense oligonucleotides and ribozymes, please see, Opalinska, J. B. and A. M. Gewirtz, Nat. Rev. Drug Discov., 2002, 1(7):503-514).

In yet an additional aspect, the present invention relates to a DNA construct comprising;

a first segment which is a regulatory region of the mouse PSP94 gene, and;

a second segment which is a gene able to be transcribed into a polynucleotide selected from the group consisting of an antisense (messenger) RNA and a ribozyme, said polynucleotide targeting a gene capable of initiating (promoting, sustaining) tumor formation.

In another aspect, the present invention relates to a transgenic non-human mammal, susceptible to prostate tumor formation, having genomically-integrated in non-human mammal cells, a DNA construct comprising:

a first segment which is a regulatory region of the mouse PSP94 gene, and;

a second segment which is a gene capable of initiating tumor formation, such that said non-human mammal expresses a tumor related agent (mainly) in the prostate (cells) of said non-human mammal inititating tumor formation.

In a further aspect, the present invention relates to a transgenic non-human mammal having genomically-integrated in non-human mammal cells, a DNA construct comprising:

a first segment which is a regulatory region of the mouse PSP94 gene, and;

a second segment which is a gene capable of initiating tumor formation such that said non-human mammal expresses said tumor gene in the prostate of said non-human mammal for initiating tumor formation.

In accordance with the present invention, the DNA construct may be, for example, selected from the group consisting of the 183 transgene, the 186 transgene, (functional) analogues and homologues thereof.

In yet a further aspect, the present invention relates to a transgenic non-human mammal having genomically-integrated in non-human mammal cells, a DNA construct as defined herein, such as, for example, a DNA construct comprising a regulatory region of the mouse PSP94 gene operatively linked to a gene encoding a therapeutic protein, a gene encoding a reporter protein, a gene encoding a (prostate) tumor-associated antigen, an antisense RNA or a ribozyme.

In accordance with the present invention, the regulatory region of the mouse PSP94 gene may be selected from the group consisting of the DNA sequence set forth in SEQ ID NO: 1, the DNA sequence set forth in SEQ ID NO: 2, the regulatory region of the 183 transgene, the regulatory region of the 186 transgene, (functional) analogues and homologues thereof.

In accordance with the present invention, the gene capable of initiating tumor formation may be a SV40 T antigen such as, for example, the SV40 large T antigen.

In accordance with the present invention, the transgenic non-human mammal may be, for example, a transgenic mouse. Further in accordance with the present invention, the non-human mammal of the present invention may also be, for example, without being limited to a dog, a cat, a monkey (of any desired species), a sheep, a cow, a pig, a horse.

In accordance with the present invention a gene capable of initiating (promoting, inducing) tumor formation may be, for example, an oncogene. Any oncogene or effective sequence thereof can be used to produce the transgenic mouse of the invention. Table 1 below, lists some known viral and cellular oncogenes, many of which are homologous to DNA sequence endogenous to mice and/or humans, as indicated. The term “oncogene” encompasses both the viral sequences and the homologous endogenous sequences. Any other oncogene, that are not part of table 1 may be suitable for the present invention (e.g., Bc12, mutated p53, cb1, etc.), more particularly in the generation of transgenic non-human mammals. Those are especially of use when studying prostate tumor development and metastasis in a transgenic non-human mammal.

In an additional aspect, the present invention relates to a DNA construct comprising a homologue of a regulatory region of the human PSP94 gene operatively linked to a gene which is able to be transcribed into an antisense messenger RNA which will render inactive a messenger RNA expressed from a gene capable of initiating tumor formation. Regulatory regions of human PSP94 gene are known in the art (Ochai, Y., et al., J. Biochem., 1995, 117(2):346-352, Nolet, S., et al., Biochem. Biophys. Acta, 1991, 1089(2):247-249). Any construct comprising a homologue of a regulatory region of the human PSP94 gene may find utility in the research and therapy directed toward prostate cancer development. As described for the mouse counterpart, genes (operatively) linked to human PSP94 regulatory regions may be, for example, without being restricted to, a gene encoding a therapeutic protein, a reporter protein, an antigen, a costimulatory molecule, an antisense and a ribozyme.

Thus in a further aspect, the present invention relates to a DNA construct comprising a homologue of a regulatory region of the human PSP94 gene, operatively linked to a gene encoding a reporter protein that may be selected, for example, from the group consisting of beta-galactosidase, luciferase, red fluorescent protein, green fluorescent protein, alkaline phosphatase, chloramphenicol acetyl transferase, and horseradish peroxidase.

In yet a further aspect, the present invention relates to a DNA construct comprising a homologue of a regulatory region of the human PSP94 gene operatively linked to a gene which is able to be transcribed into a polynucleotide selected from the group consisting of an antisense (messenger) RNA and a ribozyme, said polynucleotide targeting a gene capable of initiating (promoting, sustaining) tumor formation e.g., an antisense messenger RNA which will render inactive a messenger RNA expressed from a gene capable of initiating tumor formation.

Unless otherwise indicated, the recombinant DNA techniques utilized in the present invention are standard procedures, known to those skilled in the art. Example of such techniques are explained in the literature in sources such as J. Perbal, A Practical Guide to Molecular Cloning, John Wiley and Sons (1984), J. Sambrook et al Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present) and are incorporated herein by reference.

Those skilled in the art of molecular cloning will know that new DNA construct(s) can be made from known DNA sequence by deleting or recovering some DNA fragments using restriction endonuclease (i.e., enzyme) that will specifically recognize a DNA sequence comprised in the desired gene or DNA sequence. For example, using a KpnI (i.e., K) enzyme, any double stranded DNA comprising the sequence recognized by this enzyme (i.e., 5′-GGTACC-3′ (coding sequence shown only)) will be cut using suitable conditions. When using HindIII (i.e., H) enzyme, any double stranded DNA comprising the sequence recognized by this enzyme (i.e., 5′-AAGCTT-3′ (coding sequence shown only)) will be cut using suitable conditions. When using BamHI (i.e., B) enzyme, any double stranded DNA comprising the sequence recognized by this enzyme (i.e., 5′-GGATCC-3′ (coding sequence shown only)) will be cut using suitable conditions. When using BglII (i.e., Bg) enzyme, any double stranded DNA comprising the sequence recognized by this enzyme (i.e., 5′-AGATCT-3′ (coding sequence shown only)) will be cut using suitable conditions. When using EcoRV enzyme, any double stranded DNA comprising the sequence recognized by this enzyme (i.e., 5′-GATATC-3′ (coding sequence shown only)) will be cut using suitable conditions (i.e., suitable buffer, temperature and volume described by the manufacturer). When using PstI (i.e., P) enzyme, any double stranded DNA comprising the sequence recognized by this enzyme (i.e., 5′-CTGCAG-3′ (coding sequence shown only)) will be cut using suitable conditions. When using SalI enzyme, any double stranded DNA comprising the sequence recognized by this enzyme (i.e., 5′-GTCGAC-3′ (coding sequence shown only)) will be cut using suitable conditions. When using StuI enzyme, any double stranded DNA comprising the sequence recognized by this enzyme (i.e., 5′-AGGCCT-3′ (coding sequence shown only)) will be cut using suitable conditions. When using XbaI enzyme, any double stranded DNA comprising the sequence recognized by this enzyme (i.e., 5′-TCTAGA-3′ (coding sequence shown only)) will be cut using suitable conditions. When using HincII enzyme, any double stranded DNA comprising the sequence recognized by this enzyme (i.e., 5′-GT-pyrimidine-purine-AC-3′ (coding sequence shown only)) will be cut using suitable conditions. When using ClaI enzyme, any double stranded DNA comprising the sequence recognized by this enzyme (i.e., 5′-ATCGAT-3′ (coding sequence shown only)) will be cut using suitable conditions. When using EcoRI enzyme, any double stranded DNA comprising the sequence recognized by this enzyme (i.e., 5′-GAATTC-3′ (coding sequence shown only)) will be cut using suitable conditions. When using BsaAI enzyme, any double stranded DNA comprising the sequence recognized by this enzyme (i.e., 5′-pyrimidine-ACGT-purine-3′ (coding sequence shown only)) will be cut using suitable conditions.

Suitable conditions for restriction enzymes include, for example, suitable buffer, temperature and volume. Suitable conditions are described by manufacturers (e.g., New England Biolab, Pharmacia).

Those skilled in molecular cloning will also know that following digestion with restriction enzymes, the desired DNA may be ligated, for example, into a linearized plasmid (i.e., vector, DNA construct) or to another linear DNA molecule having matching ends. Alternatively, following digestion with restriction enzymes, the cohesive ends of DNA may be transformed to blunt ends using any suitable DNA Polymerase (e.g., T4 DNA Polymerase) or any suitable Nucleases (e.g., Mung Bean Nuclease) and the desired DNA may be ligated, for example, into a linearized plasmid (i.e., vector) or to another linear DNA molecule having suitable ends (e.g. blunt ends). Ligases (e.g., T4 DNA Ligase) will catalyze the formation of a phosphodiester bond between juxtaposed 5′ phosphate and 3′ hydroxyl termini in duplex DNA or RNA. This enzyme (when used in suitable conditions described by the manufacturer) will join blunt end and cohesive end termini as well as repair single stranded nicks in duplex DNA, RNA, or DNA/RNA hybrids.

In order for ligation to occur, the DNA molecules (e.g., desired DNA, linearized plasmid) the 5′ end of the DNA molecule must be phosphorylated. Such phosphorylation may occur for example by using Polynucleotide kinase. Suitable Polynucleotide kinase (e.g., T4 Polynuleotiude kinase) will catalyze (when used in suitable conditions described by the manufacturer) the transfer and exchange of P₁ (i.e., inorgaic phosphorus) from the gama position of ATP (i.e., adenosine triphosphate) to the 5′ hydroxyl terminus of polynucleotides (double- and single-stranded DNA and RNA) and nucleoside 3′-monophosphates.

Following ligation, DNA may be transformed in bacteria for amplification, and may be purified from lysed bacteria. Following purification, the DNA construct (linearized or not) may be transferred (e.g. transfected, transformed, electroporated, micro-injected, lipofected etc.) into a desired host (e.g., a eukaryotic cell, a oocyte, an embryonic cell, a bacteria, a yeast, etc.)

As used herein the expression “DNA construct” includes without limitation; a vector, a plasmid (e.g., linearized or not) and a DNA fragment that can be used to transfer DNA sequences from one organism to another.

As used herein the expression “PSP94 gene” relates to coding and non-coding regions of said gene.

As used herein, the expression “vector” refers to an autonomously replicating DNA or RNA molecule into which foreign DNA or RNA fragments are inserted and then propagated in a host cell for either expression or amplification of the foreign DNA or RNA molecule. The term “vector” comprises and is not limited to a plasmid, (e.g., linearized or not), or a DNA construct that can be used to transfer DNA sequences from one organism to another. The term “vector” includes viral and non-viral vector. Viral vetors may be derived, for example, from a retrovirus, a herpes virus, an adenovirus, an adeno-associated virus, Sindbis virus, poxvirus. Non-viral vector includes, but are not limited to, bacterial plasmids.

As used herein the term “transgene” refers to a DNA construct (e.g., DNA fragment) that has been incorporated into the genome of an organism.

As used herein the expression “operatively linked” refers to two or more distinguishable DNA sequences of a transgene which are linked according to recombinant technology techniques so that they may act together to control and express a protein encoded RNA in a suitable tissue or cell type. An example would be the operatively linking of a promoter/tissue-specific enhancer to a DNA sequence coding for the desired protein(s) so as to permit and control expression of the DNA sequence and the production of the encoded protein(s).

As used herein the term “regulatory region(s)” refers to region having an effect on the transcriptional control of a gene, the level of expression of a gene or on its specific expression in a given cell type or tissue type. The term “regulatory region(s)” includes tissue specific elements, promoter, enhancer, polyadenylation signal, or any regions of a gene, either upstream (5′) or downstream (3′) in either coding or non-coding region (exon or intron) having an influence on the transcriptional control of a gene or on the level of expression of a gene. The “regulatory region(s)” can be isolated from existing DNA sequence(s) or can be man-made by known techniques of molecular biology. Existing DNA sequence can be derived, for example, without being limited to, from virus, bacteria, yeast, or higher eukaryotes.

Transcription control sequences are sequences, which control the initiation, elongation, and termination of transcription. Particularly important transcription control sequences are those which control transcription initiation, such as, but not limited to, promoter, enhancer, operator and repressor sequences.

It is to be understood herein that a gene is transcribed (expressed) into a messenger RNA (spliced or unspliced). In turn a mRNA (spliced when required) is translated (expressed) into a protein.

As used herein the term “polynucleotide” refers to any polyribonucleotide or polydeoxyribonucleotide, which may be unmodified RNA or DNA, or modified RNA or DNA. “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus “polynucleotide” embraces chemically, enzymatically or metabolically modified forms of polynucleotides as typically found in nature, as well as the chemical forms of DNA and RNA characteristic of viruses and cells. “Polynucleotide” includes but is not limited to linear and end-closed molecules. “Polynucleotide” also embraces relatively short polynucleotides, often referred to as oligonucleotides.

As used herein, the term “tumor” relates to solid or non-solid tumors, metastatic or non-metastatic tumors, tumors of different tissue origin including, but not limited to, tumors originating in the liver, lung, brain, lymph node, bone marrow, adrenal gland, breast, colon, pancreas, prostate, stomach, or reproductive tract (cervix, ovaries, endometrium etc.). The term “tumor” as used herein, refers also to all neoplastic cell growth and proliferation, whether malignant or benign, and all pre-cancerous and cancerous cells and tissues.

It is to be understood herein that a polynucleotide or polynucleotide region that has a certain percentage (for example 75%, 80%, 85%, 90% or 95%) of sequence identity (homology) to another sequence may function in an equivalent or sufficient manner. A certain percentage (for example 75%, 80%, 85%, 90% or 95%) of sequence identity to another sequence (over the active region of said sequence) means that, when aligned, that percentage of bases (in the active region) is the same in comparing the two sequences. This alignment and the percent homology or sequence identity can be determined using software programs known in the art, for example, those described in Current Protocols in Molecular Biology (Ausubel et al., eds, 1987) supp. 30, section 7.7.18, Table 7.7.1. A preferred alignment program is ALIGN Plus (Scientific and Educational Software, Pennsylvania). Thus any DNA construct having significant homology to regulatory regions described herein are encompassed by the present invention.

It is to be understood herein, that if a “range”, “group of substances” or particular characteristic (e.g., temperature, concentration, time and the like) is mentioned, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example,

with respect to a temperature greater than 100° C., this is to be understood as specifically incorporating herein each and every individual temperature state, as well as sub-range, above 100° C., such as for example 101° C., 105° C. and up, 110° C. and up, 115° C. and up, 110 to 135° C., 115° C. to 135° C., 102° C. to 150° C., up to 210° C., etc.;

with respect to a temperature lower than 100° C., this is to be understood as specifically incorporating herein each and every individual temperature state, as well as sub-range, below 100° C., such as for example 15° C. and up, 15° C. to 40° C, 65° C. to 95° C., 95° C. and lower, etc.;

with respect to reaction time, a time of 1 minute or more is to be understood as specifically incorporating herein each and every individual time, as well as sub-range, above 1 minute, such as for example 1 minute, 3 to 15 minutes, 1 minute to 20 hours, 1 to 3 hours, 16 hours, 3 hours to 20 hours etc.;

TABLE 1

| Abbreviation | Virus |
|---|---|
| src | Rous Sarcoma Virus (Chicken) |
| yes | Y73 Sarcoma Virus (Chicken) |
| fps | Fujinami Sarcoma Virus (Chicken, Cat) |
| abl | Abelson Murine Leukemia Virus (Mouse) |
| ros | Rochester-2 Sarcoma Virus (Chicken) |
| fgr | Gardner-Rasheed Feline Sarcoma Virus (Cat) |
| erbB | Avian Erythroblastosis Virus (Chicken) |
| fms | McDonough Feline Sarcoma Virus (Cat) |
| mos | Moloney Murine Sarcoma Virus (Mouse) |
| raf | 3611 Murine Sarcoma.sup. + Virus (Mouse) |
| Ha-ras-1 | Harvey Murine Sarcoma Virus (Rat) |
| | Balb/c mouse; 2 loci |
| Ki-ras 2 | Kirsten Murine Sarcoma Virus (Rat) |
| Ki-ras 1 | Kirsten Murine Sarcoma Virus (Rat) |
| myc | Avian MC29 Myelocytomatosis Virus (Chicken) |
| myt | Avian Myelo Blastomas (Chicken) |
| fos | FBJ Osteosarcoma Virus (Mouse) |
| ski | Avian SKV T10 Virus (Chicken) |
| rel | Reticuloendotheliosis Virus (Turkey) |
| sis | Simian Sarcoma Virus (Woolly Monkey) |
| N-myc | Neuroblastomas (Human) |
| N-ras | Neuroblastoma, Leukemia Sarcoma Virus (Human) |
| Blym | Bursal Lymphomas (Chicken) |
| mam | Mammary Carcionoma (Human) |
| neu | Neuro, Glioblastoma (Rat) |
| ertAl | Chicken AEV (Chicken) |
| ra-ras | Rasheed Sarcoma Virus (Rat) |
| mnt-myc | Carcinoma Virus MH2 (Chicken) |
| myc | Myelocytomatosis OK10 (Chicken) |
| myb-ets | Avian myeloblastosis/erythroblastosis Virus |
| | E26 (Chicken) |
| raf-2 | 3611-MSV (Mouse) |
| raf-1 | 3611-MSV (Mouse) |
| Ha-ras-2 | Ki-MSV (Rat) |
| erbB | Erythroblastosis virus (Chicken) |

### BRIEF DESCRIPTION OF THE DRAWINGS

FIG. 1A is a schematic of DNA constructs including a 3,842 bp 2,137 bp, 1,075 bp, 761 bp, 158 bp promoter/enhancer region of mouse PSP94 gene linked to the Lac Z reporter gene.

FIG. 1B is a schematic showing the generation of DNA constructs including a mouse PSP94 promoter/enhancer region coupled with the complete exon 1 or part of intron 1 and linked to the Lac Z reporter gene.

FIG. 1C. is a schematic showing the construction of transgene 183. PSP94 promoter/enhancer region was linked with SV40Tag (both large T and small t) gene. The whole transgene is contained in a 6.4 kb Xba 1 fragment. Two internal Pst1 (P) sites are indicated. Location of primer pairs for identification of transgenic mice having the 183 transgene is indicated (Pr36 and PsSVtag).

FIG. 1D. is a schematic showing the construction of transgene 186. Part of intron 1 sequences were included as described in FIG. 1B. The linker sequence of 3′-end of intron 1 and the Lac Z (β-galactosidase) coding region is indicated at the bottom, which contains the restriction sites HindIII (exon 2 of PSP94) ClaI-HincII/StuI. The StuI site of SV40 Tag is 27 bp upstream of the first Met codon of Tag that is followed by two other Met codons. The SV40 Tag sequence is shown in small capital letters and the reading frame is also indicated by codons. Intron and exon sequences are indicated by white and black bars respectively. The whole transgene is contained in a 8.2 kb Xba1 fragment. Three Pst1 (P) sites are indicated. The location of primer pairs for identification of transgenic mice having the 186 transgene is indicated (Pr70, PsSVtag).

FIG. 1E shows a picture of a Southern blot performed on the DNA extracted from the cells of mice of lines A, B and C compared to a known copy number of the SV40 Tag gene.

FIG. 2A is a graph showing prostate targeting in transgenic mouse of line A. Results are expressed as the percentage of male (M) or female (F) mice expressing the transgene relative to the total number transgenic mice tested (M+F). Both male and female transgenic mice were assessed for non-prostate targeting (NPT)(in line A, n=116).

FIG. 2B is a graph showing prostate targeting in transgenic mouse of line B. Results are expressed as the percentage of male (M) or female (F) mice expressing the transgene relative to the total number transgenic mice tested (M+F). Both male and female transgenic mice were assessed for non-prostate targeting (NPT)(in line B, n=84).

FIG. 2C is a graph showing prostate targeting in transgenic mouse of line C. Results are expressed as the percentage of male (M) or female (F) mice expressing the transgene relative to the total number transgenic mice tested (M+F). Both male and female transgenic mice were assessed for non-prostate targeting (NPT)(in line C, n=116).

FIG. 2D is a diagram showing the disappearance of the transgene (transgene 186) in non-prostate tissue among male (M) or female (F) mice in F1, F2 and F3 generations of line C. Results are expressed as the percentage of male or female mice having the transgene in non-prostatic tissue to the total number of mice analyzed (males+females). n=number of NPT (non-prostate targeting) mice, shown by white bars over the total number of mice (male+female) tested. The percentage of HC mice (mice of line C having a high copy number of transgene 186) is shown by black bars. The total number of mice tested in F1 is 25. The total number of mice tested in F2 is 108. The total number of mice tested in F3 is 41.

FIG. 3a represents a picture of a histological section of low-grade intraepithelial neoplasia prostate samples in a PSP-TGMAP mouse (line C, 12 weeks of age). Section was stained with hematoxylin/eosin (H&E). A magnification of 40× is shown.

FIG. 3b represents a picture of a histological section showing immunohistochemistry results detecting SV40 Tag of low-grade intraepithelial neoplasia in prostate samples from a PSP-TGMAP mouse (line C, 12 weeks of age). The slide used in this picture represents a serial slide of FIG. 3a. Section was stained with hematoxylin. A magnification of 40× is shown.

FIG. 3c represents a picture of a histological section of high-grade intraepithelial neoplasia in prostate samples in a PSP-TGMAP mouse (line C, 13 weeks of age). Section was stained with hematoxylin/eosin. A magnification of 40× is shown.

FIG. 3d represents a picture of a histological section showing immunohistochemistry results detecting SV40 Tag of high-grade intraepithelial neoplasia in prostate samples from a PSP-TGMAP mouse (line C, 13 weeks of age). The slide used in this picture represents a serial slide of FIG. 3c. Section was stained with hematoxylin. A magnification of 40× is shown.

FIG. 3e represents a picture of a histological section of well differentiated adenocarcinoma arising from a high-grade intraepithelial neoplasia with micro-invasion (shown by arrows) of prostate sample in a PSP-TGMAP mouse (line C, 24 weeks of age). Section was stained with hematoxylin/eosin. A magnification of 10× is shown.

FIG. 3f represents a picture of a histological section showing immunohistochemistry results detecting SV40 Tag of well differentiated adenocarcinoma arising from a high-grade intraepithelial neoplasia with micro-invasion of prostate sample in a PSP-TGMAP mouse (line C, 24 weeks of age). The slide used in this picture represents a serial slide of FIG. 3e. Section was stained with hematoxylin. A magnification of 10× is shown.

FIG. 3g represents a picture of a histological section of well differentiated adenocarcinoma with a cribriform pattern in prostate sample of a PSP-TGMAP mouse (line C, 28 weeks of age). Section was stained with hematoxylin/eosin. A magnification of 10× is shown.

FIG. 3h represents a picture of a histological section showing immunohistochemistry results detecting SV40 Tag of well differentiated adenocarcinoma with a cribriform pattern in prostate sample of a PSP-TGMAP mouse (line C, 28 weeks of age). The slide used in this picture represents a serial slide of FIG. 3g. Section was stained with hematoxylin. A magnification of 25× is shown.

FIG. 3i represents a picture of a histological section of moderately differentiated adenocarcinoma in prostate sample of a PSP-TGMAP mouse (line A, 20 weeks of age). Arrows point at apoptotic bodies. Section was stained with hematoxylin/eosin. A magnification of 25× is shown.

FIG. 3j represents a picture of a histological section showing immunohistochemistry results detecting SV40 Tag in moderately differentiated adenocarcinoma in prostate sample of PSP-TGMAP mouse (line A, 20 weeks of age). The slide used in this picture represents a serial slide of FIG. 3i. Section was stained with hematoxylin. A magnification of 25× is shown.

FIG. 3k represents a picture of a histological section of poorly differentiated adenocarcinoma in prostate sample of a PSP-TGMAP mouse (line A, 16 weeks of age). Section was stained with hematoxylin/eosin. A magnification of 25× is shown.

FIG. 3l represents a picture of a histological section showing immunohistochemistry results detecting SV40 Tag in poorly differentiated adenocarcinoma in prostate sample of a PSP-TGMAP mouse (line A, 16 weeks of age). The slide used in this picture represents a serial slide of FIG. 3k. Section was stained with hematoxylin. A magnification of 25× is shown.

FIG. 4A is a diagram illustrating the relationship between the pathogenesis (hyperplasia (Hyp), low-grade prostate intraepithelial neoplasia (LGPIN), high-grade prostate intraepithelial neoplasia (HGPIN)), CaP (including well, moderately and poorly differentiated carcinoma) and age (16 to 19 weeks old, 20 to 23 weeks old, 24 to 27 weeks old, 28 to 31 weeks old and older than 32 weeks) of PSP-TGMAP transgenic mice of line A. N, indicates the number of mice in each age group tested.

FIG. 4B is a diagram illustrating the relationship between the pathogenesis (hyperplasia (Hyp), low-grade prostate intraepithelial neoplasia (LGPIN), high-grade prostate intraepithelial neoplasia (HGPIN)), CaP (including well, moderately and poorly differentiated carcinoma) and age (12 to 15 weeks old, 16 to 19 weeks old, 20 to 23 weeks old, 24 to 27 weeks old, 28 to 31 weeks old and older than 32 weeks) of PSP-TGMAP transgenic mice of line B. N, indicates number of mice in each age group tested.

FIG. 4C. is a diagram illustrating the relationship between the pathogenesis (hyperplasia (Hyp), low-grade prostate intraepithelial neoplasia (LGPIN), high-grade prostate intraepithelial neoplasia (HGPIN)), CaP (including well, moderately and poorly differentiated carcinoma) and age (12 to 15 weeks old, 16 to 19 weeks old, 20 to 23 weeks old, 24 to 27 weeks old, 28 to 31 weeks old and older than 32 weeks) of PSP-TGMAP transgenic mice of line C. N, indicates the number of mice in each age group tested.

FIG. 4D. is a picture of a 1.5% agarose gel showing results of RT-PCR analysis of the dorsolateral prostate tissue samples from transgenic mice, after electrophoresis of amplified DNA. Homogeneous tissue samples were selected at necropsy of different tumor grades (hyperplasia (Hyp), low-grade prostate intraepithelial neoplasia (LGPIN), well differentiated adenocarcinoma (WDCaP), and poorly differentiated CaP (PDCaP)).

FIG. 5a is a picture of gross pathological dissection of a transgenic mouse of line A at 16 weeks of age. Arrows show the location of bladder as well as a large sized (2.5 cm³) prostate cancer (CaP).

FIG. 5b is a picture of gross pathological dissection of the transgenic mouse illustrated in FIG. 5a showing enlarged renal lymph nodes (LN) after removal of the large prostate cancer mass.

FIG. 5c is a picture of gross pathological dissection of a littermate of the transgenic mouse illustrated in FIG. 5a, showing a fair sized prostate (indicated by arrow) after one month of castration.

FIG. 5D is a picture of Western blot illustrating the expression of the SV40 Tag protein in several tissue of a transgenic mouse of line A at 16 weeks of age, detected using a chemoluminescent substrate. The upper panel of FIG. 5D represents results obtained using the monoclonal antibody directed at the SV40 Tag followed by a secondary anti mouse-IgG antibody. The lower panel of FIG. 5D represents results obtained using the secondary anti mouse-IgG antibody only.

FIG. 6a represents a picture of histology sections showing immunohistochemistry results detecting p53 in high-grade prostate intraepithelial neoplasia in PSP-TGMAP mouse tissue of line A. Section was stained with hematoxylin/eosin. A magnification of 10× is shown.

FIG. 6b represents a picture of histology sections showing immunohistochemistry results detecting p53 in poorly differentiated CaP in PSP-TGMAP mouse tissue of line A. Section was stained with hematoxylin. A magnification of 10× is shown.

FIG. 6c represents a picture of histology sections showing a lymph node from PSP-TGMAP mouse tissue of line A at 16 weeks of age. Section was stained with hematoxylin/eosin. A magnification of 10× is shown.

FIG. 6d represents a picture of histology sections showing immunohistochemistry results detecting SV40 Tag in PSP-TGMAP mouse tissue of line A at 16 weeks of age. The slide used in this picture represents a serial slide of FIG. 6c. Section was stained with hematoxylin. A magnification of 10× is shown.

FIG. 6e represents a picture of histology sections showing the prostate of a PSP-TGMAP mouse tissue of line A, 2 weeks after castration. Section was stained with hematoxylin/eosin. A magnification of 10× is shown.

FIG. 6f represents a picture of histology sections showing immunohistochemistry (IHC) results detecting SV40 Tag in PSP-TGMAP mouse tissue of line A, 2 weeks after castration. The slide used in this picture represents a serial slide of FIG. 6e. Section was stained with hematoxylin. A magnification of 10× is shown.

FIG. 6g represents a picture of histology sections showing the prostate in PSP-TGMAP mouse tissue of line A, 5 weeks after castration. Section was stained with hematoxylin/eosin. A magnification of 10× is shown.

FIG. 6h represents a picture of histology sections showing higher magnification of a section of FIG. 6g demonstrating immunohistochemistry results detecting SV40 Tag expression in PSP-TGMAP mouse tissue of line C, 5 weeks after castration. Section was stained with hematoxylin. A magnification of 25× is shown.

FIG. 6i represents a picture of histology sections showing the prostate in PSP-TGMAP mouse tissue of line C, 5 weeks after castration. Section was stained with hematoxylin/eosin. A magnification of 4× is shown.

FIG. 6j represents a picture of histology sections showing negaive immunohistochemistry results detecting SV40 Tag in PSP-TGMAP mouse tissue of line C, 5 weeks after castration. The slide used in this picture represents a serial slide of FIG. 6i. Section was stained with hematoxylin. A magnification of 10× is shown.

### DETAILED DESCRIPTION OF THE INVENTION

The utility of the promoter/enhancer region (i.e. regulatory region) of the PSP94 gene in targeting expression of heterologous genes, to the prostate gland was analyzed. Three breeding lines (i.e., line A, line B line C) of the PSP-TGMAP (PSP94 gene driven transgenic mouse of adenocarcinoma of the prostate) model were established with constructs containing regulatory regions with or without additional exon/intron sequence of the PSP94 gene. These regulatory regions were operatively linked to the simian virus 40 (SV40) T antigen (Tag, including both the large T and small t antigens). Histopathology evaluation of tumor grade, transgene expression, tumor responsiveness to androgen deprivation and tumor metastatic potential were analyzed and reported herein. This study indicates that regulatory regions (i.e. the promoter/enhancer with exon/intron region) of the PSP94 gene efficiently target the expression of heterologous genes to prostate tissue. Furthermore, the PSP-TGMAP model provides a valuable tool for the analysis of prostate cancer development and for developing new gene therapy vector for the treatment of prostate cancer. Based on this study, mouse PSP94 promoter/enhancer (i.e., regulatory) region may be used in the generation of a “shuttle vector” fit for prostate targeting in both mouse CaP model and in human clinical trial.

### EXAMPLE 1

### Construction and Characterization of the Promoter/Enhancer Region of Mouse PSP94 in Prostate Cancer Cell Lines

A 3,842 base pair (bp) promoter/enhancer region of mouse PSP94 gene (SEQ ID NO: 1)(GenBank accession No. AF087140) was screened from a mouse 129Sv genomic library (Stratagene, CA) using a 600 bp (Pst1-Hind III) fragment upstream of the ATG. This fragment was subcloned and fully sequenced. Automatic DNA sequencing was performed on both DNA strands using an automatic DNA sequencer (ABI Model 377, Perkin Elmer).

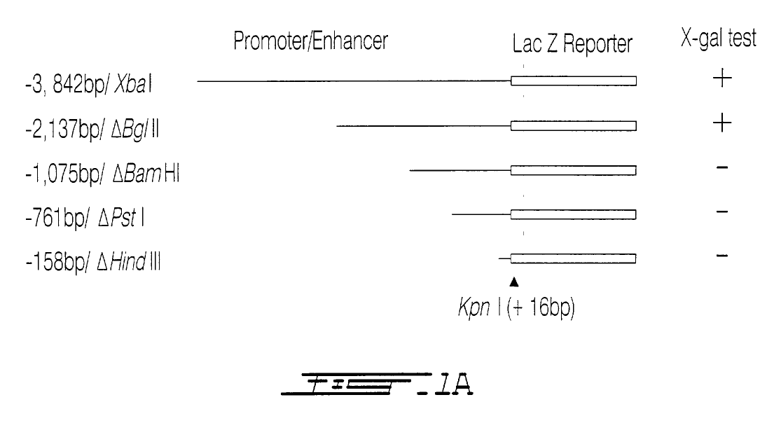

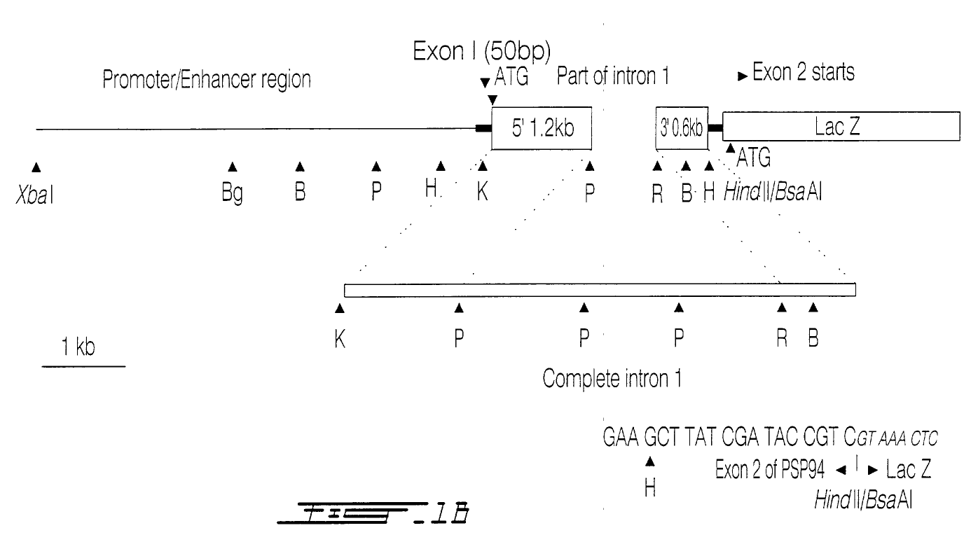

Serial progressive deletions of the mouse PSP94, 3,842 bp promoter/enhancer region were constructed using restriction sites (XbaI, BglII, BamHI, PstI and HindIII) present in this region (FIG. 1A). These constructs contained either a 2,137 bp fragment (SEQ ID NO: 2), a 1,075 bp fragment, a 761 bp fragment, or a 158 bp fragment of the promoter/enhancer region (FIG. 1A). All of the DNA constructs generated using this method were linked to the Lac Z reporter gene, encoding the beta-galactosidase (i.e., β-galactosidase or βgal) enzyme from the plasmid pSV-βgal (Promega, Toronto Canada).

FIG. 1A, shows the construction and testing of progressive deletions of a 3.842 kb promoter/enhancer region of mouse PSP94 gene linked with the Lac Z reporter gene. Positive (+) and negative (−) results are indicated on the right side. Deletion mutants are indicated by—bp numbers upstream of the first transcriptional initiation site (+1). These constructs contains the DNA sequence of the first exon going until the first Kpn I site at +16 bp. (Genebank accession no. AF033264, internet address of Genebank: www.ncbi.nlm.nih.gov. Gene bank is homed at the National Center for Biotechnology Information (NCBI), which is a division of the National Library of Medicine (NLM) at the National Institutes of Health (NIH), United States.).

FIG. 1B shows construction of both promoter/enhancer region and part of intron 1 sequences with a reporter gene Lac Z. The wild type intron 1 structure was compared with part of intron 1 subcloned in this construction. The linker sequence of the 3′-end of intron 1 and Lac Z (β-galactosidase) coding region is indicated at the bottom of the figure and contains the restriction sites: Hind III (exon 2 of PSP94)-ClaI-HincII/Bsa AI. BsaA1 site of Lac Z DNA sequence located at 7 codons upstream of the third possible translational initiation Met codon. The Lac Z DNA sequence is listed in small capitals and the reading frame is also indicated by codons (last line). B:BamH1, Bg:BglII, H:Hind III, K:Kpn1, P:Pst1, R:EcoRI. The sequence of mouse PSP94 exon 1 and intron 1 can be found in Genebank (Genebank accession no. AF033264). The sequence of mouse PSP94 exon 2 can be found in Genebank (Genebank accession no. AF033265). The sequence of mouse PSP94 promoter can be found in Genebank (Genebank accession no. AF087140).

The DNA construct containing the 3,842 bp fragment (SEQ ID NO: 1), the 2,137 bp fragment (SEQ ID NO: 2), the 1,075 bp fragment, the 761 bp fragment, and the 158 bp fragment of PSP94 promoter/enhancer region fused with a reporter gene (Lac Z) were tested in human prostate cancer cells. All of these plasmid constructs were transiently transfected (e.g., lipofected according to the manufacturer's (Invitrogen) instructions) into the LNCaP (ATCC no. CRL-1740 or CRL-10995), a human prostate cancer cell line, with a second DNA construct encoding the mouse androgen receptor (AR) under the control of the cytomegalovirus promoter. The positive control used in this experiment was cells transfected with pSV-βgal, the negative controls were cells transfected with no DNA, or cells transfected with mouse AR cDNA construct alone. Transfectants were stained with Xgal. A positive expression of the β-galacosidase enzyme in LNCaP cells is indicated by a + sign under the “X-gal test” column of FIG. 1A. The transient expression was performed by co-transfecting a DNA construct encoding the mouse AR cDNA driven by the cytomegalovirus promoter, at twice the dose of the transgene constructs.

Results shown in FIG. 1A, indicate that the 3,842 bp (SEQ ID NO: 1) and 2,137 bp (SEQ ID NO: 2) fragment of PSP94 promoter/enhancer region were able to direct Lac Z expression in LNCaP transient transfection experiments. However, the 1,075 bp, 761 bp, and 158 bp fragment of PSP94 promoter/enhancer region were not able to give detectable levels of βgal expression. These results were reproducible even if the transfection efficiency was very low (i.e., in the order of about 1/50 to 1/100 of the frequency of the positive control). The same construct were tested in human androgen independent CaP cell lines (DU145 and PC3). However, in these cell lines, the transfection frequency was even lower.

Considering the possibility that intron sequence can enhance the expression of transgenes as well as previous reports on the prostate tissue specific alternative splicing, new construct containing parts of intron sequence 1 of PSP94 were generated (Palmiter, R. D. et al., Proc. Natl. Acad. Sci. U.S.A., 88:478-482, 1991; Xuan, J. W., et al., Oncogene, 11:1041-1047, 1995).

In order to test if intron 1 sequence, the long (˜6 kilobase (kb)) intron closest to PSP94 promoter/enhancer region could increase the transfection frequency, a new DNA construct was generated (Xuan, J. W., et al., DNA Cell Biol., 18:11-26, 1999). Northern blotting experiments with multiple mouse tissue total RNA samples were performed with whole intron 1 probe (Xuan, J. W., et al., DNA Cell Biol., 18:11-26, 1999) to confirm that intron 1 does not encode any other structural protein (data not shown). FIG. 1B shows the DNA construct containing the promoter/enhancer region coupled to intron and exon sequences. This DNA construct was originally generated by a multi-step cloning procedure. It contains a 3,842 bp promoter/region, a complete exon 1 sequence followed by a 1,100 bp fragment of the 5′ region of intron 1 and a 600 bp fragment of the 3′ region of intron 1 as well as part of exon 2. Part of exon 2 of PSP94 has been linked in the same reading frame as Lac Z coding region (near the last ATG). Exon 2 sequence of the PSP94 gene was maintained in the same reading frame as the Lac Z gene during the cloning procedures. This new promoter was linked to the Lac Z gene coding region. Every construct described or illustrated herein may be generated by more simple approaches (e.g., PCR) since the DNA sequence of PSP94 gene (including promoter, exon and intron) and cDNA sequence are available from Genebank (see: Genebank Accesion no. AF033264, AF033265, AF033266, AF087140, and U89840).

Transfection to LNCaP cells was performed in 6 well plates using Lipofectin reagent (Gibco/BRL, Burlington, Ontario, Canada), according to the manufacturer's instructions. Approximately 10 μg of plasmid DNA were used for 3×10⁵ cells. A positive control pSV-βgal plasmid was used to monitor the efficiency of transfection. Positive transfectant cells were detected by a standard X-gal staining procedure. The pSV-βgal transfected cells usually showed 10% of the cell population positive for β-galactosidase expression. Each transfecting plasmid was co-transfected with a mouse androgen receptor (AR) encoding plasmid (i.e., a CMV5-mAR, where the AR expression is driven by the cytomegalovirus promoter) in a 1:2 ratio. Positive transfectant cells were detected by a standard X-gal staining procedure. Results of in vitro test in LNCaP cells with this new construct were still positive, with slightly higher frequencies.

### EXAMPLE 2

### Construction of Transgenes Comprising Regulatory Regions of Mouse PSP94 Gene and Generation of Transgenic Mice

Based on the results of in vitro tissue cell culture analysis, the 3,842 bp PSP94 promoter/enhancer, was selected for the generation of transgenic mice. Additional construct were however generated in order to increase the level of expression of the SV40 TAg. Compared with reporter genes (e.g., Chloramphenicol acetyl transferase (CAT), Lac Z), detection of SV40 Tag has proved to be very accurate and sensitive, and is very easy to detect in a large amount of mouse population by anatomical analysis at necropsy.

Two transgenes (the 183 and 186 transgenes) were generated using regulatory regions of PSP94 and both the SV40 large T and small t antigens (from the plasmid pBSV-1, a SV40 genomic clone) cloned into a pBluescript DNA plasmid (Stratagene). The SV40 TAg gene was obtained following digestion of a subclone of pBSV-1 using the Stu I and Xho I restriction enzymes, generating a 2.7 kb fragment.

FIG. 1C and FIG. 1D show the construction of transgene 183 or 186 respectively. The 183 transgene promoter comprises essentially the 3,842 base pair promoter described in FIG. 1A. The 186 transgene promoter comprises essentially the promoter described in FIG. 1B. Briefly, the PSP94 promoter/enhancer region was linked with SV40 Tag (both large T and small t) gene. The whole 183 transgene is contained in a 6.4 kb Xba 1 fragment where two internal Pst1 (P) sites are indicated (Xuan, J. W. et al., DNA Cell Biol. 18:11-26, 1999).

Locations of primer pairs for identification of transgenic mice are also indicated (Pr36, PsSVtag, Pr70). Part of intron 1 sequences were subcloned as described in FIG. 1B. The linker sequence of 3′-end of intron 1 and Lac Z (β-galactosidase) coding region is indicated on the bottom of FIG. 1D, which contains the restriction sites Hind III (exon 2 of PSP94) Cla1-HincII/Stu 1. StuI site of SV40Tag is 27 bp upstream of the first Met codon of Tag and followed by another two Met codons (reading frame organized in codons). Intron and exon sequences are indicated by white and black bars respectively. The whole 186 transgene is contained in a 8.2 kb Xba 1 fragment. Three Pst1 (P) sites are indicated. The 186 transgene is illustrated in FIG. 1D. This construct was generated using a multi-step cloning procedure. It contains a 3,842 bp promoter/enhancer region, exon 1, part of intron 1 (a Kpn1-Pst1 1.5 kb fragment and an EcoR1-Hind III fragment) and part of exon 2 linked to the SV40 Tag gene.

A 6.5 kb fragment of the 183 transgene or a 8.2 kb fragment of the 186 transgene was obtained following digestion using the XbaI enzyme. These fragments were purified with an EluTip kit (Mandel, toronto) for micro-injection into fertilized mouse eggs. The identity of both transgenes, especially the junctional area between the PSP94 regulatory regions and the SV40 Tag region, have been confirmed by DNA sequencing analysis. The predicted nucleotide sequence of both transgenes regulatory regions correspond to the published sequence (based on the PSP94 gene, see GeneBank accession no. AF033264, AF033265, AF033266, AF087140, and U89840). Generation of transgenic mice, including micro-injection into fertilized mouse eggs [C57BL/6 x CBA] F1 hybrids, was performed according to standard procedures.

There was no lethal effect of the PSP-TGMAP transgene in embryo development, as in the course of breeding, there were almost equal male (446) and female (411) progenies obtained. Candidate transgenic mice were first identified by a quick PCR test using chromosomal DNA extracted from toe cells. DNA was extracted by digesting tissue samples in PCR buffer containing proteinase K (100 μg/ml) and a nonionic detergent at 55° C. for 3 hours. The crude chromosomal DNA was denatured by boiling and subjected to PCR reaction using primer pairs illustrated by horizontal arrows in FIG. 1C (Primer Pr36 sequence is as follow: 5′-GGC AAC AGC GTG TCA AAG-3′ (SEQ ID NO: 3), primer mPr70 sequence is as follow: 5′-GCC TTA GTC TCT GAT TGC TC-3′ (SEQ ID NO: 4), primer PsSVTag sequence is as follow: 5′-CAA GAC CTA GAA GGT CCA TTA GC-3′ (SEQ ID NO: 5). After weaning, tail DNA was purified using the EASY kit (Quiagen, CA) and the PCR reaction was repeated.

Breeding of transgenic mice was carried out first by mating with wild type ([C57BL/6 x CBA] F1 hybrids) then gradually by mating in between transgenic mice identified by PCR and Southern blotting experiments.

After breeding with wild type ([C57BL/6 x CBA] F1 hybrids, three transgenic founder breeding lines (F0 of A, B, C lines), out of 18 transgenic mice, were characterized and successfully established. The A line was generated from transgene 183 (named 183-2), and the B and C lines were generated from transgene 186 (named 186-3, 186-9 respectively).

The transgene copy number in the three transgenic mouse founder breeding lines (F0 of A, B and C lines) and of the F1, F2, F3 progenies were characterized by Southern blotting experiment. FIG. 1E, shows the result of this Southern blotting analysis of three transgenic breeding mice lines: A (183) and B (186-3), C (186-9). About 5 μg of transgenic mouse tail chromosomal DNA for each lane was digested by Pst1 restriction enzyme and loaded onto a 0.5% agarose gel. A full length SV40 Tag DNA fragment (2.7 kb) labeled by ³²P dCTP was used as a probe. For semi-quantitative determination and comparison of the copy number of the transgene in three mouse breeding lines, known amount of SV40 Tag fragment DNA (from 0-500 relative copies as indicated) comparable to the copy number of mouse chromosomal DNA per lane were loaded (left). Higher copies of the transgene in line C (186-9) were possibly arrayed from either of two directions of the transgene. High intensity of the signal in the Southern blot must be related to repeated copy of the transgene, while the integrated chromosomal flanking fragments (only one copy) are not visible in this blot.

Results shown in FIG. 1E, indicate that the transgenic mouse of line A has approximately 100 copies (per mouse genome) of the transgene integrated into its genome. The transgenic mouse of line B has a slightly higher copy number (i.e., approximately 150 copies per genome). The transgenice line C founder mouse (186-9) and some offsprings (F1, F2, F3) possess 5-10 times more copies (approximately 500 copies/per genome). Transmittance of this higher copy (HC) transgene in total breeding offsprings is with a ratio (HC/Total) of 5/25 (20%) in Fl, and 6/108 (5.5%) in F2, and 0/41 in F3 respectively, indicating a transmittance mechanism of non-sex chromosomal linkage, non-Mendelian segregation. The non-Mendelian segregation of PSP-TGMAP C-line of the founder mouse (186-9) may indicate that the extraordinarily higher copy number of the transgene is possibly unstable in the transmittance of transgenic material to the next generation.

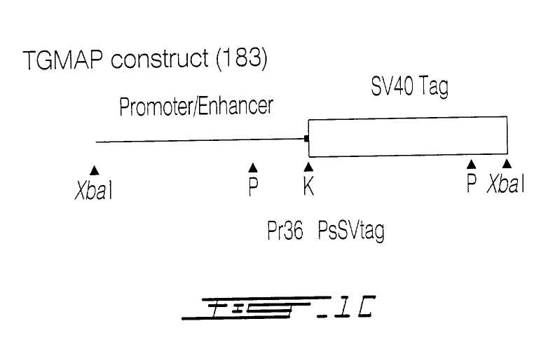

Results presented in FIG. 2 are graphs showing prostate targeting in the three transgenic mouse lines (FIG. 1A: line A, FIG. 1B: line B, FIG. 1C: line C) and is expressed relative to the percentage of total transgenic mice tested. Both male (M) and female (F) transgenic mice were assessed for non-prostate targeting (NPT) in these experiments. FIG. 1D shows the elimination of NPT of Line C in the process of breeding in three generations (F1, F2, F3). N indicates total numbers of mice detected in each line.

Targeting of the transgene expression in three transgenic breeding lines (a total of 210 males and 164 females) to the prostate was evaluated. First, autopsy studies of mice in breeding line A (183) and B (186-2) were performed and showed that the SV40 Tag expression induced morphological and histological changes detectable only in VP (ventral prostate) lobes and DLP (dorsolateral prostate) lobes, while non-prostate targeting (NPT) including seminal vesicle (SV) and coagulation gland (CG), was not found. These results were observed for a total population of 116 mice (64 males, 52 females) of line A, at 16 weeks and up to one year of age (FIG. 2A). For line B a total population of 84 mice (47 males, 37 females) was observed at 12 weeks and up to one year of age (FIG. 2B).

Founder mouse F0 (186-9) of line C (FIG. 2C) showing an extraordinary high number of copies of the transgene was the only mouse founder showing non-prostate targeting (NPT) in lymph node (autopsy at 23 weeks of age), in addition to the prostate tissue specific changes. These results were demonstrated by Western blotting experiments, using an ECL kit (enhanced chemiluminescent kit, from Amersham, Oakville, Ont) (data not shown). In brief, fresh dissected prostate lobes (˜0.2 g) were homogenized in 1 ml ice-cold 0.01 M phosphate buffered saline (PBS) and then centrifuged at 10,000× g. The proteins found in the clarified cell lysates were analyzed using a 15% sodium dodecyl sulfate-polyacrylamide gel electrophoresis (SDS-PAGE) followed by Western blot. Horseradish peroxidase (HRP) conjugated goat anti-mouse IgG was used as a secondary antibody (Dimension Laboratories, Mississauga, ON).

Results illustrated in FIG. 2C and FIG. 2D, indicate that although the overall NPT record in mice of line C is 4.3% (9 out of a total of 210 (including F0, F1, F2 and F3)), in F1 progenies of line C the NPT record was 20% (1 male, 4 females out of 25 F1) in such tissues as skin, liver, intestine and bone. The frequencies of NPT appearance in F2 mice progenies decreased significantly to 3.7% (3 males, 1 female out of 108 F2). In F3, no NPT was found in 3 breeding families of 41 offsprings. These results indicate that NPT is decreased and eliminated from F0 to F3 in mice of line C.

A correlation of NPT with the transmittance of high copy (HC) of the transgene was also found (FIG. 2D), because the rate of HC mice also decreased accordingly from F0 to F3, and the majority of NPT mice are HC mice (6/11). Total number (male+female) of NPT/HC mice in F1, F2, and F3 were 5/3, 4/3 and 0/0 respectively. The proportions of homozygous (mating in between C mice) over heterozygous (mating between mice of line C and wild type) in the resultant progenies were not changed from F2 (4/6 families) to F3 offspring (2/3 families). These results indicate that the tumor induced will not be affected by mating either with wild type or between PSP-TGMAP mice. The PSP94 promoter used in this study is strong enough to induce prostate cancer in herozygous and homozygous mice.

### EXAMPLE 3

### Histopatholgical Characterization of the Prostate Tumor Development in in the Three PSP-TGMAP Breeding Lines

The anatomy of the prostate complexe and male accessory gland (i.e., the ventral (VP) and the dorsolateral (DLP) prostate lobes, the coagulation gland (CG, or anterial gland: AP) the seminal vesicles (SV)), of the transgenic mice was observed (Imasato, Y., et al., Endocrinol., 142:2138-2146, 2001). All tissue samples were subjected to gross pathological, surgical and anatomical detection, and any suspect abnormal tissue samples were taken immediately for processing histology slide. Non-prostate targeting (NPT) record means the presence of tumor(s) in non-prostate tissue, detected by procedures listed herein, whith no prostate neoplastic changes. For detection of macro- or micro-metastasis, tissue samples collected at necropsy or autopsy were divided in two parts. One part was fixed in PBS-formalin for hematoxylin and eosin (H&E) staining or for immunohistochemistry studies. The other part was used for total cellular RNA or protein extraction using the Trizol reagent (Lifetechnologies) as discussed below.

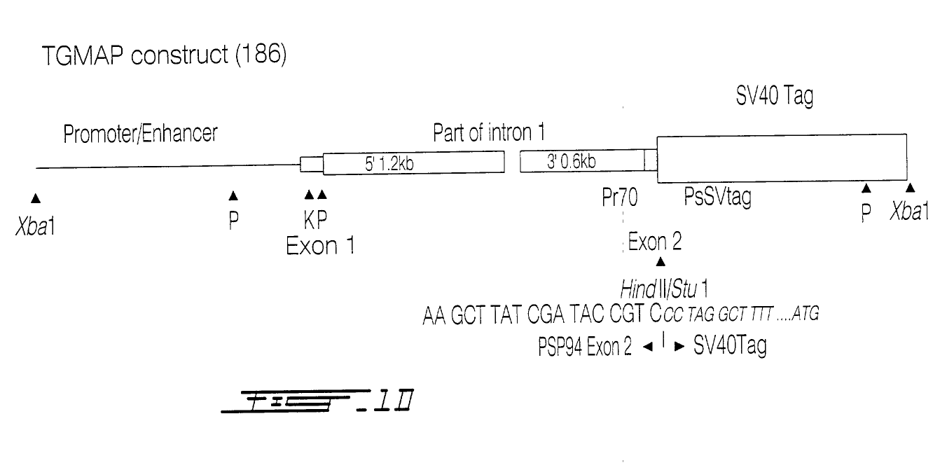

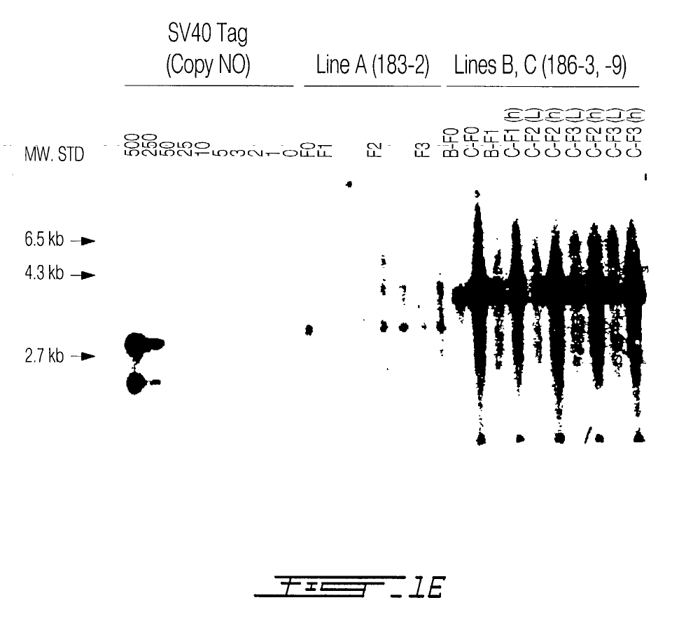

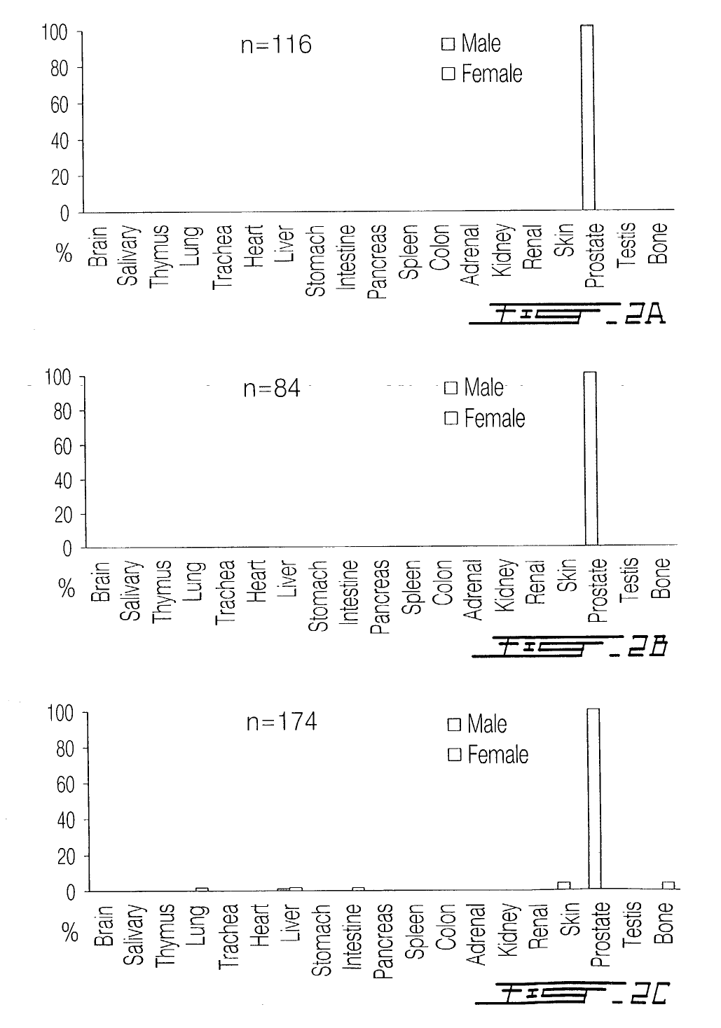

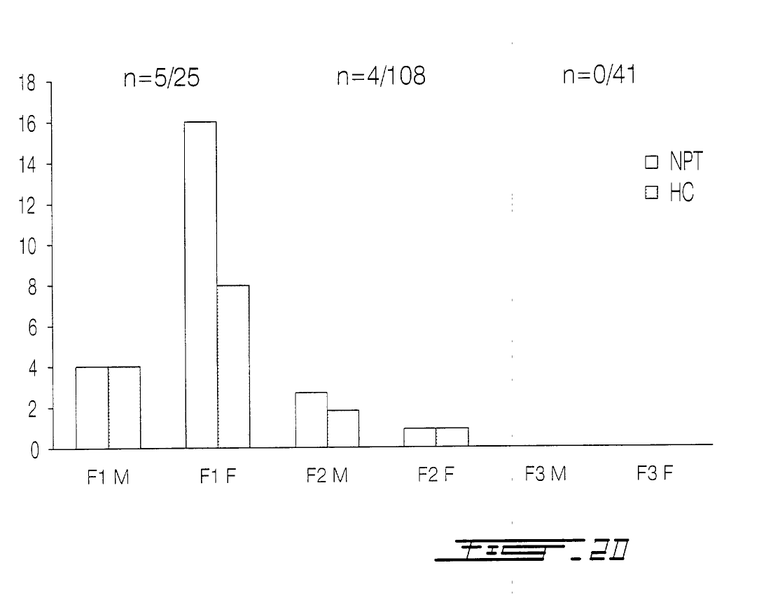

Targeting of transgene expression to the prostate of 210 transgenic male mice from the three transgenic breeding lines (line A, line B, line C) was demonstrated by histopathological studies. Normal VP and DLP lobes (FIG. 3a) were composed of acinic glands lined by columnar secretory epithelial cells with round oval nuclei and pale cytoplasm. No abnormalities were seen in the prostates of the normal control (n=5, FIG. 3a). To study tumor development in PSP-TGMAP model, transgenic mice were sacrificed at different ages (i.e., between 8 to 38 weeks of age).

According to the diagnostic criteria set up in the human CaP and the basic conceptual similarity, the neoplastic changes in the prostates of the three breeding lines of the transgenic mice (line A, line B, line C) were classified by the following six grading categories:

[1] Hyperplasia (Hyp) show circumscribed epithelial proliferation with round, small and uniform nuclei.

[2] Low-grade prostate intraepithelial neoplasia (LGPIN: FIG. 3a, FIG. 3b) shows increased focal cellularity with mild enlarged nuclei of variable size and shape, increased chromatin density and scant cytoplasm (FIG. 3a).

[3] High-grade prostate intraepithelial neoplasia (HGPIN: FIG. 3c, FIG. 3d) is featured by marked nuclear atypia, more hyperchromatic and chromatin clumping than the LGPIN, increased mitotic and apoptotic rates and increased cellularity with stratification pattern.

[4] Well differentiated adenocarcinoma (WDCaP: FIG. 3e, FIG. 3f, FIG. 3g, FIG. 3h) is characterized by several patterns: microinvasion identified as microacinar glands at the base of HGPIN glands compressing the surrounding stroma (FIG. 3e); invasive glandular differentiation pattern (FIG. 3f) and multiple histological patterns including papillary, tufting, cribriform patterns (FIG. 3g).

[5] Moderately differentiated adenocarcinoma (MDCaP: FIG. 3i, FIG. 3j) shows some acini completely filled with malignant cells and more frequent appearance of apoptotic bodies, but still with the glandular structure (FIG. 3i).

[6] Poorly differentiated carcinoma (PDCaP: FIG. 3m, FIG. 3n) contains sheets of malignant cells with no glandular features (FIG. 3m).

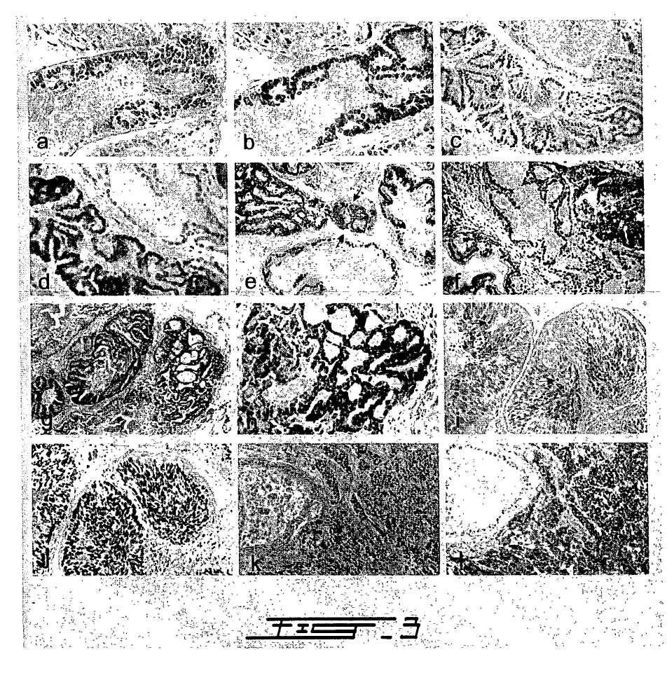

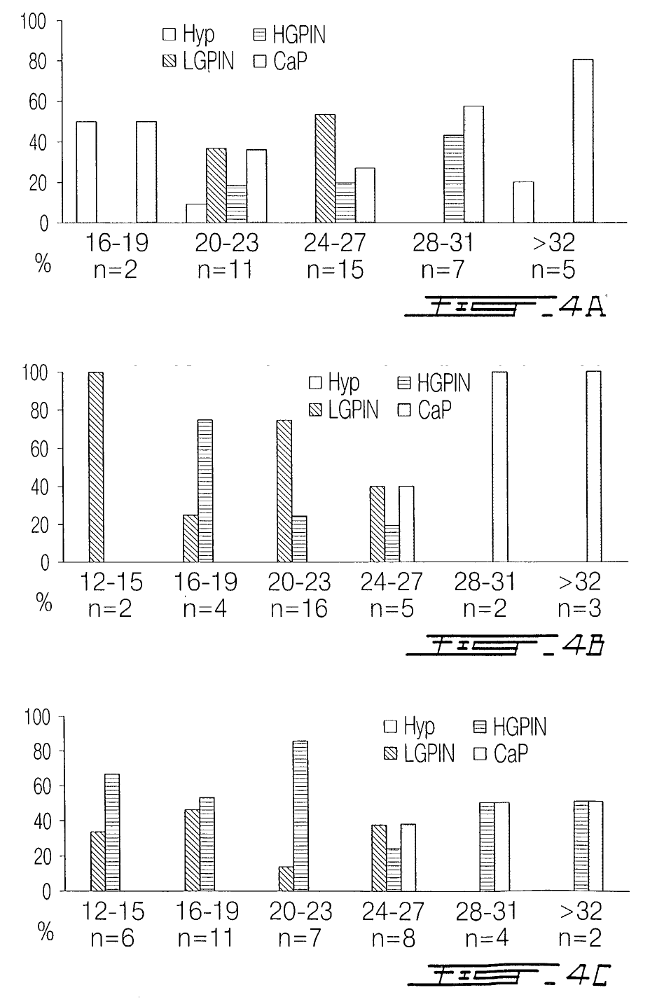

Correlation between the age and histopathological changes is summarized in FIG. 4. Hyperplasia (Hyp) was detected by 10 weeks of age in the three lines (A, B, C). A few mice of line A showed an unexpected appearance of Hyp at 16 to 32 weeks of age.

By 12 weeks of age, all mice of line B and line C (in line A it started by 16 weeks of age) showed changes associated with prostate intraepithelial neoplasia (i.e., including LGPIN and HGPIN). All three lines showed a correlation of PIN with age. Low-grade prostate intraepithelial neoplasia was found in 44% (11 mice out of 25) of mice in the three lines at age 12-19 weeks. This number then decreased gradually through the age groups and PIN was no longer found at 28 weeks of age or older (FIG. 4). High-grade prostate intraepithelial neoplasia started as early as 12 weeks of age in mice of line C (4 mice out of 6), and was detected in 48% of all three lines of mice at 12 to 19 weeks of age (12 mice out of 25). By 24 weeks, some mice of lines B and C with HGPIN, showed micro-invasion to the stroma (FIG. 3g).

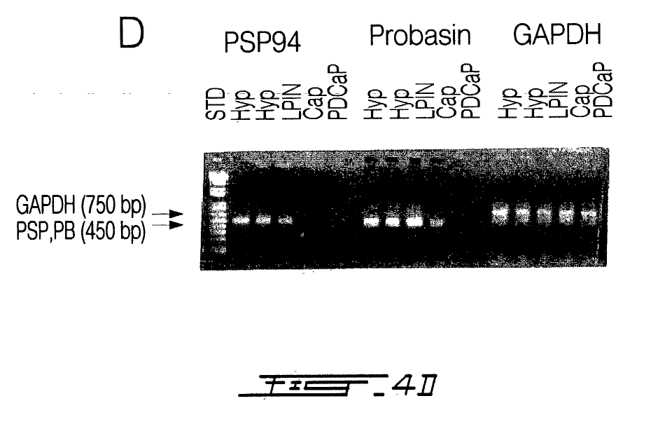

Well differentiated adenocarcinoma started by 24 weeks of age in all three lines, mostly in ventral and dorsolateral the prostate lobes (not in coagulation gland or seminal vesicle). It was detected in 13% (2 mice out of 15), 40% (2 mice out of 5), 25% (2 mice out of 8) of lines A, B and C respectively. By 32 weeks of age or later, WDCaP was detected in 40%, 100% and 50% of lines A, B, and C respectively. Moderately differentiated adenocarcinoma was mainly detected in mice of line A by 16 to 19 weeks of age. Poorly differentiated carcinoma started as early as 16 weeks of age in mice of line A (10 mice out of 40). Eight mice out of ten with PDCaP showed large visible tumors (FIG. 5a), and the rest (2 mice out of 10) had no visible tumor. PDCaP was detected in one mouse of line C, by 28 weeks of age (1 mouse out of 4).

The neoplastic changes induced by SV40 Tag expression was targeted specifically in the prostate in PSP-TGMAP mice and temporally occurred (10 weeks) in a prostate specific mode as well (i.e. correlating with male puberty and sexual maturity).

The experimental CaP in PSP-TGMAP model has shown some predictable features of tumor development. All mice of lines A, B and C will have hyperplastic changes by 10 weeks of age. By 12 weeks, 100% of mice in line B and C will develop intraepithelial neoplasia transformation (i.e., LGPIN or HGPIN) detected by IHC of SV40 Tag expression. Most mice of line A will develop intraepithelial neoplasia changes in the prostate after 20 weeks associated with IHC-detectable SV40 Tag signal (FIG. 4) and will show faster tumor growth (i.e., tumors of 2 to 3 cm³ of size) as early as 19 weeks of age (10 mice out 40). The PSP-TGMAP model illustrated here has been characterized and demonstrated by several experiments performed in this study; [1] PSP-TGMAP model is shown to be an experimental metastasis model with distant metastatic deposits. Mice (n=8) showing metastasis were characterized first by gross pathology detection (large palpable PDCaP with apparently enlarged renal lymph nodes), by IHC and Western blotting analysis of SV40 Tag expression in those metastatic tissues. NPT was characterized for tumor formation in non-prostate tissues with the prostate tissue able to evolve to HGPIN changes without metastatic potential. [2] Castration test of PSP-TGMAP mice showed the prostatic tissue specific responsiveness to androgen deprivation, however some tumors were refractory to castration. [3] Correlation of SV40 Tag with p53 expression in normal LGPIN, HGPIN and the three carcinoma grades was also observed.

### EXAMPLE 4

### Correlation of Sv40 Tag Expression and Tumor Progression in the Three PSP-TGMAP Breeding Lines

In order to determine the correlation between the histopathology results described in example 3 and the expression of the SV40 Tag, experiments of immunohistochemistry and RT-PCR were performed on prostate cells. Briefly, monoclonal antibodies against SV40 Tag (Calbiochem, CA) and p53 (AB-1, Oncogene, Cambridge, Mass.) were used for immunohistochemistry (IHC) study of mouse tissue samples using an ABC kit (StreptABC complex kit, DAKO, Mississauga, Ont). The antigen-antibody binding was detected using 3,3, diaminobenzidine/hydrogen peroxide as a substrate. Briefly, deparaffinized, and rehydrated sections were treated with 0.3% hydrogen peroxide in methanol for 15 minutes at room temperature to block endogenous peroxidase activity. A retrieval solution (i.e., Target, DAKO) was used following autoclaving for 10 minutes at 121° C. After blocking with phosphate buffered saline (PBS) containing 10% goat serum, sections were incubated with the first antibody at room temperature for 1 hour. Dilutions of 1:100 and 1:20 were used for the SV40 Tag monoclonal antibody (2 μg/ml) and p53 antibody (5 μg/ml) respectively. A negative control was performed by replacing the primary antibody with PBS. All IHC slides were counterstained with hematoxylin.

In consistence with results of histopathology studies, the expression of SV40 Tag was detected by immunohistochemistry (IHC) in the three established transgenic mouse lines and showing a correlation with tumor progression. In the initiation of hyperplastic epithelium, there was no positive nuclear staining for the SV40 Tag oncoprotein (i.e., under the detection limit of IHC). The development of LGPIN correlated with the SV40 Tag expression in the prostate epithelium as illustrated in FIG. 3b, FIG. 3d, FIG. 3h, FIG. 3j, FIG. 3l. The SV40 Tag signal (i.e., expression) was mostly observed in the cell nuclei. The number of positive epithelial cells, as well as the intensity of the immunofluorescence signal (i.e. degree of staining or immunoreactivity) was found to be increased in HGPIN compared to LGPIN (compare FIG. 3B and FIG. 3d) in the three lines. These results suggest a correlation between the level of expression of the SV40 Tag and the pathogenesis observed. Mice of line C showed a stronger immunoreactivity in terms of numbers of immunofluorescence-positive cells as well as in term of the intensity of immunofluorescence signal, than the other two lines (lines A and B). Mice with well differentiated adenocarcinoma and poorly differentiated carcinoma showed stronger expression of the SV40 Tag than those showing LGPIN or HGPIN (FIG. 3h, FIG. 3j, FIG. 3l).

To confirm the prostate specific origin and to study gene expression in different stages of PSP-TGMAP mice, RT-PCR analysis of PSP94 and probasin gene expression was performed in dorsolateral prostate lobe samples from relatively homogenous tissues samples (n=3) with different grades of tumor. Briefly, Trizol solution (Gibco-BRL, Burlington, Ont) was used to isolate total RNA according to the manufacturer's instructions. RT-PCR was performed as previously reported (Xuan, J. W., et al., Oncogene, 11:1041-1047, 1995; Xuan, J. W., et al., DNA Cell Biol., 16:627-638, 1997). Briefly, first strand cDNA was synthesized using total RNA, an oligo-dT primer, and the MMTV reverse transcriptase (Gibco/BRL) according to manufacturer's protocol. Standard RT-PCR procedure was performed as previously reported. For amplification of mouse PSP94 and mouse probasin mRNA, specific primer pairs were used (for the mouse probasin gene; mPB-PR1: 5′-AAG ATA AAT GAA GGC TCA CCA TTG-3′ (SEQ ID NO: 6), mPB-PR2: 5′-CAT ATT GAT GTT TCA GGT TCC AGG-3′ (SEQ ID NO: 7), for the mouse PSP94; mPSP-1: 5′-CCT GTA AGG AGT CCT GCT TTG TC-3′ (SEQ ID NO: 8), mPSP-2: 5′-ATG CTG GCT CTG CCT TCT GAG T-3′ (SEQ ID NO: 9). For semi-quantitative RT-PCR, a house keeping gene; glyceraldehyde-3-phosphate dehydrogenase (GAPDH) was used as an internal control in the PCR reaction. A number of PCR reaction cycles were first tested for logarithmic amplification by comparing intensity of ethidium bromide staining of amplified cDNA fragments separated in 1.5% agarose gel electrophoresis (AGE). PCR samples (10 μl) from the GAPDH amplification (752 bp) were taken every 5 cycles up to 20 cycles during the PCR amplification procedures. For PSP94, samples were taken after 40 cycles of amplification while for mPB, samples were taken after 30 cycles of amplification. The number of cycles was chosen by the lowest cycle number giving a visible band on a 1.5% agarose gel following electrophoresis and ethidium bromide staining.

As shown in FIG. 4D, both PSP94 and mPB gene expression were eliminated in well differentiated adenocarcinoma and poorly differentiated carcinoma. PSP94 gene expression decreased earlier than mPB during tumor development.

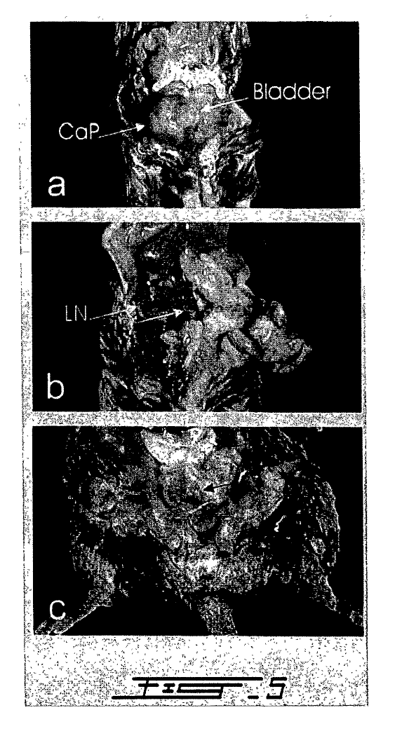

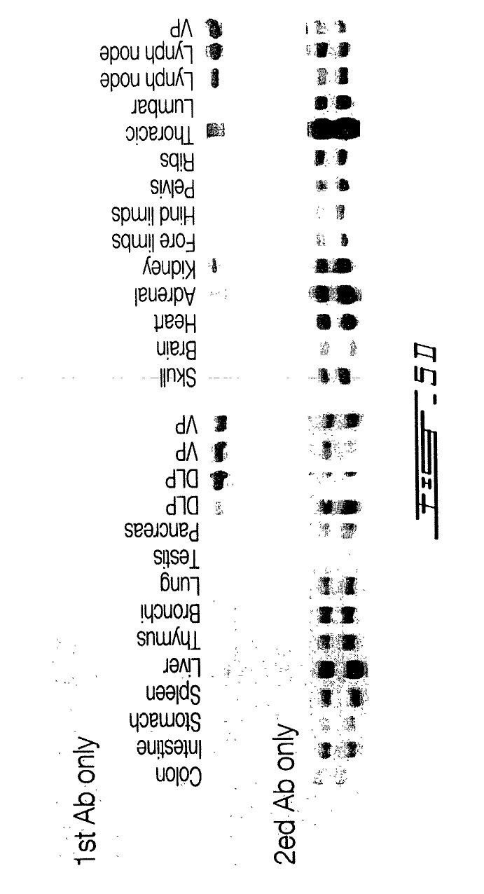

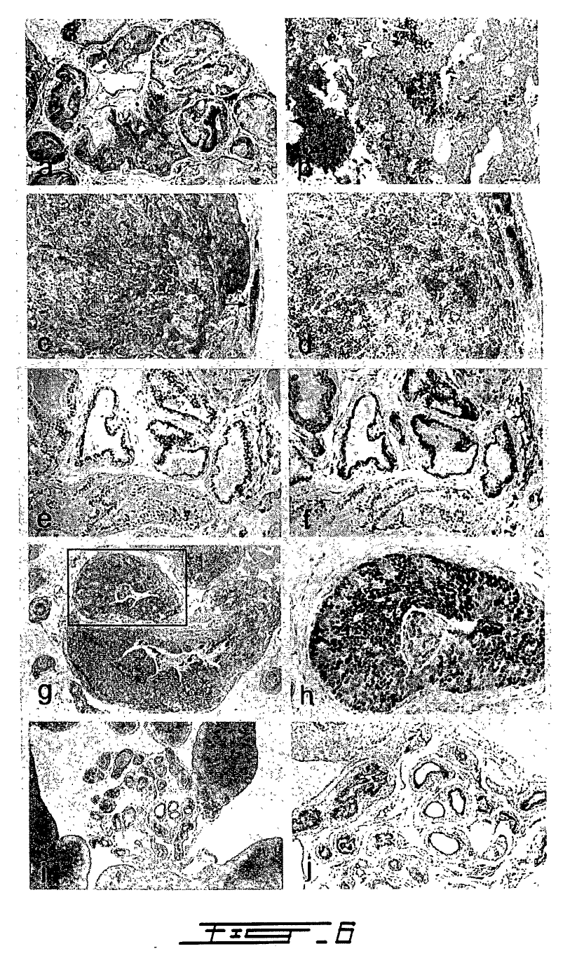

Because the SV40 Tag is known to stabilize the accumulated p53 protein in the nucleus, immunohistochemistry analysis of p53 was conducted in prostate samples from different grades of PSP-TGMAP mice (line A, n=4). Mice with LGPIN showed weak p53 signal in the nucleus (data not shown). In HGPIN this signal was stronger (i.e., more intense) and appeared only in those nuclei with typical HGPIN changes (FIG. 6a). In PDCaP samples, p53 signals were found to be strong and homogenous in isolated areas (FIG. 6b).

### EXAMPLE 5

### Analysis of Metastatic CaP in the PSP-TGMAP Model

In order to determine if the PSP-TGMAP model showed metastatic potential, three mice out of six (16 weeks of age) of line A mice showing visible (palpable tumor; FIG. 5a) prostate tumors were selected for gross pathological study. All three mice showed enlarged renal lymph nodes (shown FIG. 5b). Western blotting experiments, for detecting the SV40 Tag expression, were performed using cell lysates of biopsy samples from different tissues. As shown in FIG. 5D, metastatic SV40 Tag expression was seen in lymph nodes and in the upper pole of kidney tissues. In order to demonstrate that the metastatic cells in the lymph nodes were of prostatic origin, histological and immunohistochemistry analysis on samples of lymph nodes were performed on serial slides separately (FIG. 6d). As shown in FIG. 6c, the metastatic lesions in lymph nodes were composed of sheets of the prostate malignant cells in most of the lymph node area, which were histologically similar to the prostate PDCa malignant cells in A mice. The replacement of the lymph node cortex with undifferentiated epithelial cells was evidenced. In line B and C, high-grade prostate intraepithelial neoplasia with local invasion (WDCaP, n=4 shown in FIG. 3e) had been detected at 28 to 31 weeks of age, but no distant metastasis was found.

### EXAMPLE 6

### Responsiveness to Androgen Deprivation in Mice of the PSP-TGMAP Model

Since androgen responsiveness is a fundamental physiology of the prostate, transgenic mice of the different lines were tested and characterized by responsiveness to castration. Androgen ablation was accomplished under anesthesia by surgical castration via the scrotal route. Two groups of mice (from line A and C) at 20 to 26 weeks of age were selected; (A) was from a faster tumor growing family of A line and was tested for tumor mass shrinkage after castration; (C) was from an established prostate targeting family of C with 100% IHC immunoreactivity toward the SV40 Tag at 13 weeks of age. This group was tested for changes of IHC signal after castration. Two weeks after castration, the autopsy samples from the prostate (n=3) from each group were analyzed. FIG. 6e shows involution and atrophic changes in the whole prostate including areas of prostate intraepithelial neoplasia and hyperplastic acini. Immunohistochemistry analysis on a serial slide of FIG. 6e shows weak immunoreactivity toward the SV40 Tag protein (FIG. 6f) as compared with uncastrated control mice (data not shown), indicating responsiveness to androgen deprivation of PSP-TGMAP model. One month after castration, ⅓ of castrated mice of line A showed HGPIN with weak immunoreactivity, while ⅔ showed visible, fair sized carcinoma (FIG. 5c, H&E see FIG. 6g) with positive immunoreactivity (FIG. 6h). After one month of castration, mice of line C (4 mice out of 4), showed atrophic changes in the prostate gland (FIG. 6i) with a marked increase in the stroma ratio, with IHC negative immunoreactivity to the Tag oncoprotein (FIG. 6j). The results indicate that mice of line C are responding to castration (i.e., androgen deprivation) since SV40 Tag decreases and disappears.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention. Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A DNA construct comprising a regulatory region of the mouse PSP94 gene.

2. The DNA construct of claim 1, wherein said regulatory region of the mouse PSP94 gene is selected from the group consisting of the DNA sequence set forth in SEQ ID NO: 1, the DNA sequence set forth in SEQ ID NO: 2, the regulatory region of the 183 transgene, the regulatory region of the 186 transgene, analogues and homologues thereof.

3. A DNA construct comprising a regulatory region of the mouse PSP94 gene operatively linked to a gene capable of initiating tumor formation.

4. The DNA construct of claim 3, wherein said gene capable of initiating tumor formation is a SV40 T antigen.

5. The DNA construct of claim 4, wherein said SV40 T antigen is the SV40 large T antigen.

6. The DNA construct of claim 3, wherein said regulatory region of the mouse PSP94 gene is selected from the group consisting of the DNA sequence set forth in SEQ ID NO: 1, the DNA sequence set forth in SEQ ID NO: 2, the regulatory region of the 183 transgene, the regulatory region of the 186 transgene, analogues and homologues thereof.

7. A DNA construct comprising the transgene 183.

8. A DNA construct comprising the transgene 186.

9. A DNA construct comprising a regulatory region of the mouse PSP94 gene operatively linked to a gene encoding a therapeutic protein.

10. The DNA construct of claim 9, wherein said therapeutic protein is selected from the group consisting of a cytotoxic protein, a protein causing apoptosis, an anti-oncoprotein, a protease, a suicide protein, a cytokine, a chemokine, a costimulatory molecule and an antigen.

11. The DNA construct of claim 9, wherein said regulatory region of the mouse PSP94 gene is selected from the group consisting of the DNA sequence set forth in SEQ ID NO: 1, the DNA sequence set forth in SEQ ID NO: 2, the regulatory region of the 183 transgene, the regulatory region of the 186 transgene, analogues and homologues thereof.

12. The DNA construct of claim 10, wherein said suicide protein is selected from the group consisting of herpes simplex virus-1 thymidine kinase and Escherichia coli cytosine deaminase.

13. The DNA construct of claim 10, wherein said cytotoxic protein is selected from the group consisting of the A chain of diphteria toxin, ricin, and abrin.

14. The DNA construct of claim 10, wherein said protein causing apoptosis is selected from the group consisting of caspases, Fas-Ligand, Bax and TRAIL.

15. The DNA construct of claim 10, wherein said anti-oncoprotein is selected from the group consisting of p53, p21, and Rb.

16. The DNA construct of claim 10, wherein said protease is selected from the group consisting of awsin, papain, proteinase K, and carboxypeptidase.

17. The DNA construct of claim 10, wherein said cytokine is selected from the group consisting of IL-1, IL-2, IL-6, IL-12, GM-CSF, G-CSF, M-CSF, IFN-alpha, IFN-beta, IFN-gamma, TNF-alpha, and TNF-beta.

18. The DNA construct of claim 10, wherein said chemokine is selected from the group consisting of Mig-1alpha, Mig-1beta, IP-10, and MCP-1.

19. A DNA construct comprising a regulatory region of the mouse PSP94 gene operatively linked to a gene encoding a reporter protein.

20. The DNA construct of claim 19, wherein said reporter protein is selected from the group consisting of beta-galactosidase, luciferase, red fluorescent protein, green fluorescent protein, alkaline phosphatase, chloramphenicol acetyl transferase, and horseradish peroxidase.

21. The DNA construct of claim 19, wherein said regulatory region of the mouse PSP94 gene is selected from the group consisting of the DNA sequence set forth in SEQ ID NO: 1, the DNA sequence set forth in SEQ ID NO: 2, the regulatory region of the 183 transgene, the regulatory region of the 186 transgene, analogues and homologues thereof.

22. A transgenic non-human mammal, susceptible to prostate tumor formation, having genomically-integrated in non-human mammal cells, a DNA construct comprising:
a) a first segment which is a regulatory region of the mouse PSP94 gene, and;
b) a second segment which is a gene capable of initiating tumor formation.

23. The transgenic non-human mammal of claim 22, wherein said gene capable of initiating tumor formation is a SV40 T antigen.

24. The transgenic non-human mammal of claim 23, wherein said SV40 T antigen is the SV40 large T antigen.

25. The transgenic non-human mammal of claim 22, wherein said regulatory region of the mouse PSP94 gene is selected from the group consisting of the DNA sequence set forth in SEQ ID NO: 1, the DNA sequence set forth in SEQ ID NO: 2, the regulatory region of the 183 transgene, the regulatory region of the 186 transgene, analogues and homologues thereof.

26. The transgenic non-human mammal of claim 22, wherein said transgenic non-human mammal is a transgenic mouse.

27. A transgenic non-human mammal having genomically-integrated in non-human mammal cells, a DNA construct comprising:
a) a first segment which is a regulatory region of the mouse PSP94 gene, and;
b) a second segment which is a gene capable of initiating tumor formation.

28. The transgenic non-human mammal of claim 27, wherein said gene capable of initiating tumor formation is a SV40 T antigen.

29. The transgenic non-human mammal of claim 28, wherein said SV40 T antigen is the SV40 large T antigen.

30. The transgenic non-human mammal of claim 27, wherein said regulatory region of the mouse PSP94 gene is selected from the group consisting of the DNA sequence set forth in SEQ ID NO: 1, the DNA sequence set forth in SEQ ID NO: 2, the regulatory region of the 183 transgene, the regulatory region of the 186 transgene, analogues and homologues thereof.

31. The transgenic non-human mammal of claim 27, wherein said DNA construct is selected from the group consisting of the 183 transgene, the 186 transgene, analogues and homologues thereof.

32. The transgenic non-human mammal of claim 27, wherein said transgenic non-human mammal is a transgenic mouse.

33. A transgenic non-human mammal having genomically-integrated in non-human mammal cells, a DNA construct as defined in claim 9.

34. The transgenic non-human mammal of claim 33, wherein said transgenic non-human mammal is a transgenic mouse.

35. A transgenic non-human mammal having genomically-integrated in non-human mammal cells, a DNA construct as defined in claim 19.

36. The transgenic non-human mammal of claim 35, wherein said transgenic non-human mammal is a transgenic mouse.

37. A DNA construct comprising;
a) a first segment which is a regulatory region of the mouse PSP94 gene, and;
b) a second segment which is a gene able to be transcribed into a polynucleotide selected from the group consisting of an antisense RNA and a ribozyme said polynucleotide targeting a gene capable of initiating tumor formation.

38. The DNA construct of claim 37, wherein said regulatory region of the mouse PSP94 gene is selected from the group consisting of the DNA sequence set forth in SEQ ID NO: 1, the DNA sequence set forth in SEQ ID NO: 2, the regulatory region of the 183 transgene, the regulatory region of the 186 transgene, analogues and homologues thereof.

39. A DNA construct comprising a homologue of a regulatory region of the human PSP94 gene, operatively linked to a gene encoding a therapeutic protein selected from the group consisting of a cytotoxic protein, a protein causing apoptosis, an anti-oncoprotein, a protease, a suicide protein, a cytokine, a chemokine a costimulatory molecule and an antigen.

40. The DNA construct of claim 39 wherein said suicide protein is selected from the group consisting of herpes simplex virus-1 thymidine kinase and Escherichia coli cytosine deaminase.

41. The DNA construct of claim 39 wherein said cytotoxic protein is selected from the group consisting of the A chain of diphteria toxin, ricin, and abrin.

42. The DNA construct of claim 39 wherein said protein causing apoptosis is selected from the group consisting of caspases, Fas Ligand, Bax and TRAIL.

43. The DNA construct of claim 39 wherein said anti-oncoprotein is selected from the group consisting of p53, p21, and Rb.

44. The DNA construct of claim 39 wherein said protease is selected from the group consisting of awsin, papain, proteinase K, and carboxypeptidase.

45. The DNA construct of claim 39 wherein said cytokine is selected from the group consisting of IL-1, IL-2, IL-6, IL-12, GM-CSF, G-CSF, M-CSF, IFN-alpha, IFN-beta, IFN-gamma, TNF-alpha, and TNF-beta.

46. The DNA construct of claim 39 wherein said chemokine is selected from the group consisting of Mig-1alpha, Mig-1beta, IP-10, and MCP-1.

47. A DNA construct comprising a homologue of a regulatory region of the human PSP94 gene, operatively linked to a gene encoding a reporter protein selected from the group consisting of beta-galactosidase, luciferase, red fluorescent protein, green fluorescent protein, alkaline phosphatase, chloramphenicol acetyl transferase, and horseradish peroxidase.

48. A DNA construct comprising a homologue of a regulatory region of the human PSP94 gene operatively linked to a gene which is able to be transcribed into a polynucleotide selected from the group consisting of an antisense RNA and a ribozyme, said polynucleotide targeting a gene capable of initiating tumor formation.


SEQUENCE LISTING (9 sequences)

SEQ ID NO: 1 - DNA, 3842 bp, Mus musculus
  misc_feature (1)..(3842)
tctagagctcgcggccgcgagctctaatacgactcactatagggcgtcgactcgatctggagcaagtggtatagaacctacctaccaggccaggcctaagggatgccctatcattagtttccagcacacccaaagaaaggaaggcacttggactccttctttaacaaccccttgtcttaaggtgctttctactgctgtgagaaagaaccatgactgtgctggtttgaacgagaatgggcctcatgggctcctacatttgaatacttcctctgtagcggatgccatttctagtttccttcctctctcctttgtgcctgtggaccacaatgcacactcctagtgaccgttccagtaccaagcctgcctccctgtcaccatgttccctgtcatggtatcacatgaactcatccccggcagatgtaagcctataataaactttttcttccatgaattgctttggtcatggtgtcttaggataggaagataaaagtaactgagatagccctatatacaatatatgccctctatatacatatacatatatatatatgtatgtatatgtatgtgtatatatatatatatatatatatattatgcaatatgtcctctatactagaaaatattagcaatctttcccattaggacaggaatctgaagtagagagaaacctatataaataaccttgttaagccagccctgtgattcccaccgcttctcaacagaatgagccaccccacccctcagcatgtatgtagaccttgcttctttgactcttcattcctgacctgtaaacccatgccatataaacttattttagtaaatatatgtgtaattcatatttactatttaccttgtgtttatttaattactaggcccagattaaaaaccctaagctagcaatagtaaaataggctttctctctagccttgcagagacttgaagtgccggggtggggagatacccaagggagcccccatctgctcagaggagaaggggaggggggaagaattgtgggagggggatactgggaggaaggcaatgagcttgatataaagtgaataaataaaaaataattaattaattaaaaaatgctttctgtacagccatttatcagacactgagctaaatatgttagacatatttccccttatttttcatgtccatccttggaggtggggaatcaatgtctctgtttcataaacagttagacaaagaaccagaaatgttaagaaattcccccaagttcacacagctgttcaaggaaggatttggatgggagcccttggctaccccagctctccatacacctgggctacaccgatgctacattcttgctaccccaaatcacagcttgaaatcttaactcttcatgtaagggctattaaaaaggagggaaattccaggaggaagtttggtcatcggtgtagagccaccatactctaccatgtgaggatgtcaccaaagacacagccccatggcacctgggagagggccctcacaaggacacgaccacaatggcacactgatctcagacatgcagcccctcagtctgtgaaggattcattttagctatcgtctgagcatctccagtctacggcccgtgctatgccagccctaacggagggcacagatcacaccaagggagacacacaggaggagtaaggttggtgtcttcagactaaggattgtttcccccccccaataagatcttcatgtaacgtttactttacaacatttaaattagattttgcctgatgtataaaatcataaaattgtacatactaacagggaaccatgtgaagtttttttgttttttgtttttttttaagatttctttattattacatctaagtacactgtagctgtcttcattagcaccagaagagggcatcagatctcattacggatggttgtgagccaccatgtggttgctgggatttgaactcaggacctacggaagagcagtcgatgctcttaaccactgagccatctctccagccccccatgtgaagttttaacatgtttatgctgttgaagtgattcatcttcctgtcttcttcagtactgggatttcgagtgtgtgtcatcatgcccattgtgtgtgtgtgtgtgtgtgtgtgtgtgtgtgtgtgtgtaggcacatacataaatgtggagatattgtatgacactatccctggggatatatgaacacacatctactcatcccacatatggaatcaaggacagacaatataacccaaaggcagccatggcaaagctaggagtttagtggactagttatgtgagaatgggtcagggttagtacagaaacagggatggctcagaggcagtgagcggtactacaggaaaacccgcccatccaagcggggatgcaactccagccacagccctggaactctttacaggacttacagactgatcagcaggtgacaggccagtggctccaccaacagttgtcaatggcttctgtaacctcaagagttggggtggggcaggcaagtgagtcttgtcaaggttttagcttcctgagttgttcaagttcaaatactttctgattctcatgagcctcccatctggttccttaagtgaatgtttcagttgggaggaaactgctatgtatggagaggccagatagcaacctcacttgttcctcagatgccatccccactgtttttggagacagggcctgtcactggtctggggctcaccaagacttgacttgctgacctgtaggcccataggaccaaaggcccataggcccggggatccgcctgtttcagcaccactagtgctgggattgcaagcctgtcccatcatgcatagccttttgctgttaggttgtcatggttgcagccgagcactgtacgacagagctagctcttcagcctttatacaggctctggttttcgggtagttctggggatgaaacccagggcttctaacatgctagaccaggattctactaagtgagctatacccagccccaaggttcacccttttctggctgcttgtttccttgtatatacatcagctatggacacagggtgcgctccatcacgtggcgactgtgaacagcgctgcagtgaactttcaggagtgcagacggcttctgagtgggattgctggattataaattacttcaagtttttaatcattcaagggcactccatgctgtttcctatgcaggaagccactgttgaacagagaaaggaaggccttgaggaaacagagaaaagactaaatgctcaggagaatgagatagcttgagagatgtgagcatgtggcttgtcaaaagacagctaggctgtgggcatgactcatccacaccatggaaaatggctccatggactttctcatcctggaccctaaggccagcacctcctgggcctcagctccctggcctaccaggctgacctggaggctaaccttatgcctcagcacttggctgctgtgagtcccattcccgagccctcatttctttgcctatgagctcaggaaagaacattgagggttgcgtagcggtcttcccagtgcaggatgaaggctcacagggaaagctctttctgtctgctctctcccatgaagggcaacagcgtgtcaaaggtgaggaatacaccccgaactggtcaagtggccccagtgcctagaaaaccctagtgataagcacagaattggggtaaaagcttactatgcgagagacttataaagcaagagaaattataagttcctgttcagccctcagcaaatacccaatatgttcttggaacagtatcgttccaagaacacccacgagtggttatcctcacctgtataaataggaaggcagcattcatgcttgc

SEQ ID NO: 2 - DNA, 2137 bp, Mus musculus
  misc_feature (1)..(2137)
gatcttcatgtaacgtttactttacaacatttaaattagattttgcctgatgtataaaatcataaaattgtacatactaacagggaaccatgtgaagtttttttgttttttgtttttttttaagatttctttattattacatctaagtacactgtagctgtcttcattagcaccagaagagggcatcagatctcattacggatggttgtgagccaccatgtggttgctgggatttgaactcaggacctacggaagagcagtcgatgctcttaaccactgagccatctctccagccccccatgtgaagttttaacatgtttatgctgttgaagtgattcatcttcctgtcttcttcagtactgggatttcgagtgtgtgtcatcatgcccattgtgtgtgtgtgtgtgtgtgtgtgtgtgtgtgtgtgtgtaggcacatacataaatgtggagatattgtatgacactatccctggggatatatgaacacacatctactcatcccacatatggaatcaaggacagacaatataacccaaaggcagccatggcaaagctaggagtttagtggactagttatgtgagaatgggtcagggttagtacagaaacagggatggctcagaggcagtgagcggtactacaggaaaacccgcccatccaagcggggatgcaactccagccacagccctggaactctttacaggacttacagactgatcagcaggtgacaggccagtggctccaccaacagttgtcaatggcttctgtaacctcaagagttggggtggggcaggcaagtgagtcttgtcaaggttttagcttcctgagttgttcaagttcaaatactttctgattctcatgagcctcccatctggttccttaagtgaatgtttcagttgggaggaaactgctatgtatggagaggccagatagcaacctcacttgttcctcagatgccatccccactgtttttggagacagggcctgtcactggtctggggctcaccaagacttgacttgctgacctgtaggcccataggaccaaaggcccataggcccggggatccgcctgtttcagcaccactagtgctgggattgcaagcctgtcccatcatgcatagccttttgctgttaggttgtcatggttgcagccgagcactgtacgacagagctagctcttcagcctttatacaggctctggttttcgggtagttctggggatgaaacccagggcttctaacatgctagaccaggattctactaagtgagctatacccagccccaaggttcacccttttctggctgcttgtttccttgtatatacatcagctatggacacagggtgcgctccatcacgtggcgactgtgaacagcgctgcagtgaactttcaggagtgcagacggcttctgagtgggattgctggattataaattacttcaagtttttaatcattcaagggcactccatgctgtttcctatgcaggaagccactgttgaacagagaaaggaaggccttgaggaaacagagaaaagactaaatgctcaggagaatgagatagcttgagagatgtgagcatgtggcttgtcaaaagacagctaggctgtgggcatgactcatccacaccatggaaaatggctccatggactttctcatcctggaccctaaggccagcacctcctgggcctcagctccctggcctaccaggctgacctggaggctaaccttatgcctcagcacttggctgctgtgagtcccattcccgagccctcatttctttgcctatgagctcaggaaagaacattgagggttgcgtagcggtcttcccagtgcaggatgaaggctcacagggaaagctctttctgtctgctctctcccatgaagggcaacagcgtgtcaaaggtgaggaatacaccccgaactggtcaagtggccccagtgcctagaaaaccctagtgataagcacagaattggggtaaaagcttactatgcgagagacttataaagcaagagaaattataagttcctgttcagccctcagcaaatacccaatatgttcttggaacagtatcgttccaagaacacccacgagtggttatcctcacctgtataaataggaaggcagcattcatgcttgc

SEQ ID NO: 3 - DNA, 18 bp, Unknown
  Primer PR36 used in amplication of transgene
ggcaacagcgtgtcaaag

SEQ ID NO: 4 - DNA, 20 bp, Unknown
  Primer mPr70  used in amplification of transgene
gccttagtctctgattgctc

SEQ ID NO: 5 - DNA, 23 bp, Unknown
  Primer PsSVTag used in amplification of transgene
caagacctagaaggtccattagc

SEQ ID NO: 6 - DNA, 24 bp, Unknown
  Primer mPB-PR1 used in amplification of mouse probasin gene
aagataaatgaaggctcaccattg

SEQ ID NO: 7 - DNA, 24 bp, Unknown
  Primer mPB-PR2 used in amplification of mouse probasin gene
catattgatgtttcaggttccagg

SEQ ID NO: 8 - DNA, 23 bp, Unknown
  Primer mPSP-1  used in amplification of mouse PSP94 gene
cctgtaaggagtcctgctttgtc

SEQ ID NO: 9 - DNA, 22 bp, Unknown
  Primer mPSP-2 used in amplification of mouse PSP94 gene
atgctggctctgccttctgagt